(19) United States
(12) Patent Application Publication
(10) Pub. No.: US 2025/0186584 A1
(43) Pub. Date: Jun. 12, 2025

(54) METHODS OF TREATING BILIARY TRACT CANCER USING ANTI-PD-L1 ANTIBODY IN COMBINATION WITH CHEMOTHERAPY

(71) Applicant: ASTRAZENECA AB, Södertälje (SE)

(72) Inventors: Shao-chun CHANG, Wilmington, DE (US); John KURLAND, Wilmington, DE (US); Gordana VLAHOVIC, Wilmington, DE (US)

(21) Appl. No.: 18/846,025

(22) PCT Filed: Oct. 25, 2022
(86) PCT No.: PCT/EP2022/079828

§ 371 Date: Sep. 11, 2024

Related U.S. Application Data
(60) Provisional application No. 63/321,215, filed on Mar. 18, 2022.

(51) Int. Cl. A61K 39/395; A61K 31/7068; A61K 33/243; A61K 39/00; A61P 35/00

(52) U.S. Cl. CPC A61K 39/39558; A61K 31/7068; A61K 33/243; A61P 35/00; A61K 2039/505; A61K 2039/545

(57) ABSTRACT

The disclosure relates to methods for treating biliary tract cancer (BTC) using an anti-PD-L1 antibody in combination with chemotherapy.

### FIELD OF THE DISCLOSURE

The disclosure relates to methods for treating biliary tract cancer (BTC) using an anti-PD-L1 antibody in combination with chemotherapy.

### BACKGROUND

Biliary tract cancer (BTC), a heterogeneous group of malignancies that includes intrahepatic and extrahepatic cholangiocarcinoma, gallbladder cancer, and ampulla of Vater cancer, is typically diagnosed at advanced stages for which curative surgery is not feasible and prognosis is poor (Valle et al., Lancet 397 (10272): 428-44, 2021). Despite trials evaluating several targeted therapies including cediranib, erlotinib, cetuximab, panitumumab, ramucirumab, and merestinib (Valle et al., 2015; Lee et al., 2012; Malka et al., 2014; Leone et al., 2016; Valle et al., Lancet Oncol. 22 (10): 1468-82, 2021), first-line standard of care for advanced disease, gemcitabine and cisplatin chemotherapy, has remained unchanged for the past decade and is associated with a median overall survival of 11.7 months and an estimated 24-month survival rate of approximately 15% (Valle et al., 2010), highlighting the need for new therapies (Brindley et al., 2021); Boilève et al., 2021).

In addressing the need for improved methods for clinical management of BTC, the disclosure provides methods comprising administration of an anti-PD-L1 antibody in combination with chemotherapy, specifically gemcitabine and cisplatin chemotherapy, as a treatment for patients with advanced biliary tract cancer.

### SUMMARY

The disclosure provides methods for treating biliary tract cancer (BTC) using an anti-PD-L1 antibody in combination with chemotherapy.

In one aspect, the disclosure herein provides a method for increasing overall survival in a patient with biliary tract cancer (BTC), comprising treating the patient with an anti-PD-L1 antibody and chemotherapy.

In another aspect, the disclosure herein provides a method for extending progression-free survival in a patient with biliary tract cancer (BTC), comprising treating the patient with an anti-PD-L1 antibody and chemotherapy.

In a further aspect, the disclosure herein provides a method of treating a patient with biliary tract cancer (BTC), the method comprising treating the patient with an anti-PD-L1 antibody and chemotherapy.

These and other features and advantages of the present disclosure will be more fully understood from the following detailed description taken together with the accompanying claims. It is noted that the scope of the claims is defined by the recitations therein and not by the specific discussion of features and advantages set forth in the present description.

### BRIEF DESCRIPTIONS OF THE DRAWINGS

FIG. 1 shows the CONSORT Diagram.

FIG. 2 shows TOPAZ-1 study design.

FIG. 3 shows Durvalumab or placebo plus gemcitabine/cisplatin dosing schedule.

FIG. 4 shows Kaplan-Meier Curves of Overall survival and Progression-free Survival in the Full Analysis Set. CI denotes confidence interval, Durva+Gem+Cis denotes durvalumab plus gemcitabine and cisplatin, and Placebo+Gem+Cis denotes placebo plus gemcitabine and cisplatin.

FIG. 5 shows Epanechnikov Kernel-smoothed Hazard Functions.

FIG. 6 shows Forest Plots of Overall Survival and Progression-free Survival by Subgroup for Durvalumab versus Placebo in the Full Analysis Set. Size of circle is proportional to the number of events observed. Race is defined as patients who identify as Asian or non-Asian regardless of geography. Rest of the world includes patients enrolled in Europe, North America, and South America. CI denotes confidence interval, Durva+Gem+Cis denotes durvalumab plus gemcitabine and cisplatin, ECOG=Eastern Cooperative Oncology Group, PD-L1=programmed cell death ligand 1, Placebo+Gem+Cis denotes placebo plus gemcitabine and cisplatin, and TAP=tumor area positivity (proportion of tumor and/or immune cells with PD-L1 staining at any intensity).

FIG. 7 shows the primary endpoint of overall survival. Overall survival was improved by the addition of durvalumab to gemcitabine and cisplatin. The median overall survival (95% CI) was increased from 11.5 months to 12.8 months. After 6 months, the difference between the durvalumab plus gemcitabine and cisplatin and placebo plus gemcitabine and cisplatin curves grew; and the difference continued to increase as time went on. The difference in overall survival increased from 6% at 12 months, to 10% at 18 months, to 15% at 24 months.

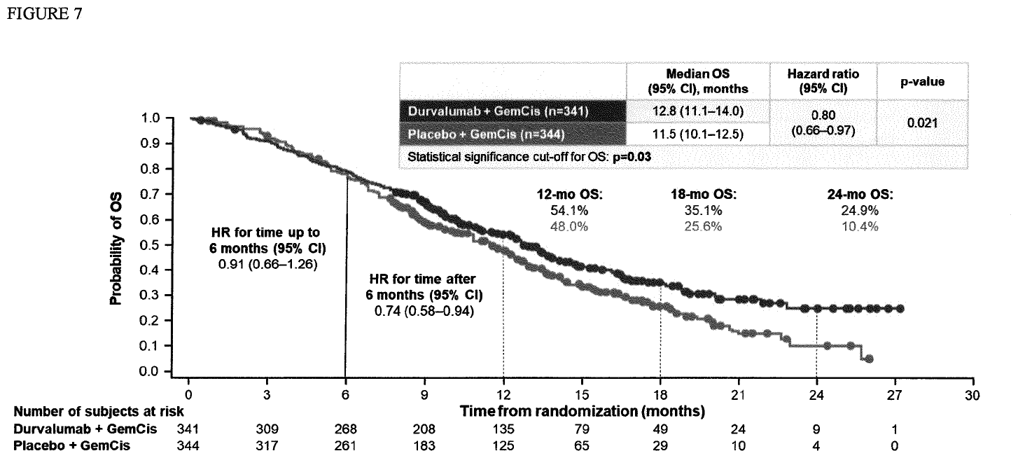

FIG. 8 shows the subgroup analysis of overall survival. The benefit of the addition of durvalumab to gemcitabine and cisplatin was observed across all subgroups.

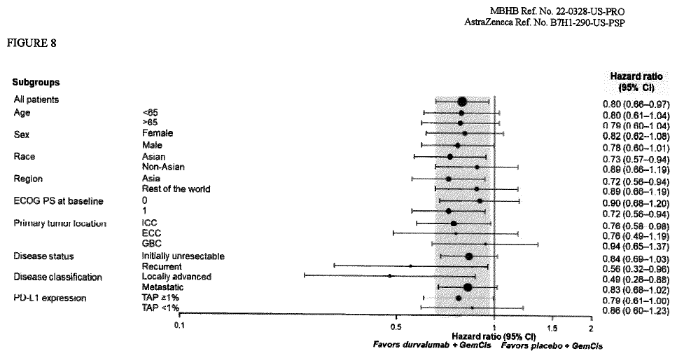

FIG. 9 shows the overall survival in subgroups by PD-L1 expression. The benefit of the addition of durvalumab to gemcitabine and cisplatin was observed across PD-L1 expression.

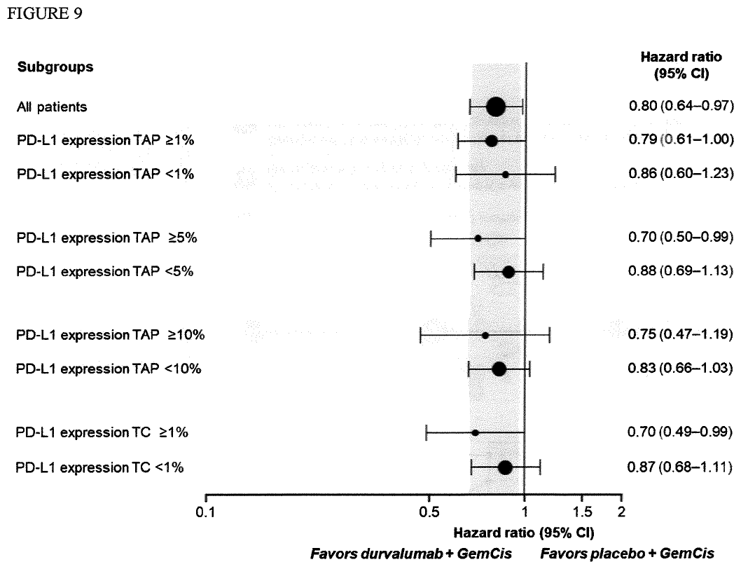

FIG. 10 shows the Tumor Area Positivity (TAP) score using the Ventana PD-L1 (SP263) Assay. PD-L1 status was tracked on tumor cells and immune cells and the TAP score was measured, which considered both tumor and immune cell PD-L1 expression. The benefit of the addition of durvalumab to gemcitabine and cisplatin was observed regardless of PD-L1 expression.

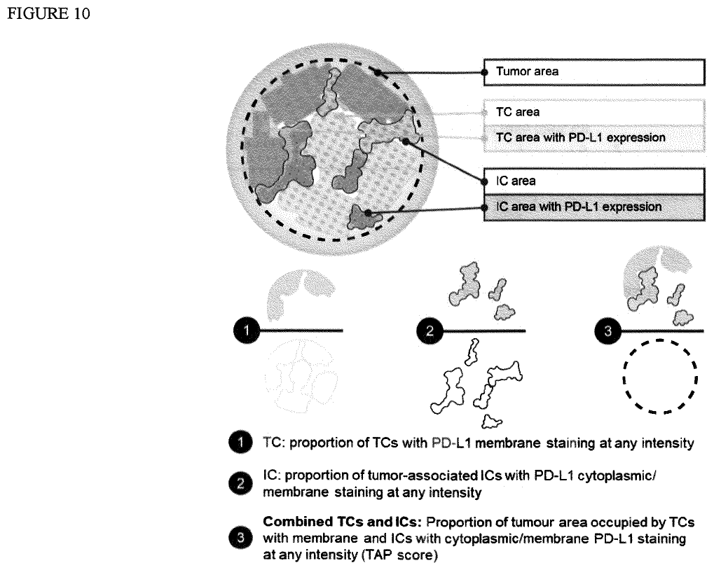

FIG. 11 shows the results for the secondary endpoint of progression-free survival (PFS). Median PFS (95% CI) was improved from 5.7 months to 7.2 months with the addition of durvalumab to gemcitabine and cisplatin.

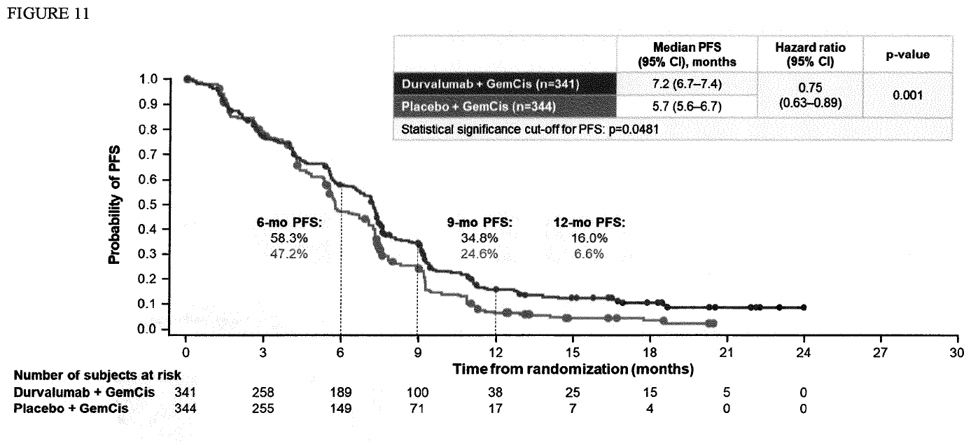

FIG. 12 shows the results for the secondary endpoints of tumor response (A) objective response rate (ORR), and (B) DoR in the tumor response for durvalumab+GemCis (durvalumab arm) and Placebo+GemCis (placebo arm). The ORR was improved from 18.7% in the placebo arm to 26.7% in the durvalumab arm. The median time to response was quicker in the durvalumab arm, 1.6 months, compared to 2.7 months in the placebo arm. More patients remained in response after 9 months, 32.6% in the durvalumab arm compared to 25.3% in the placebo arm and after 12 months, 26.1% in the durvalumab arm compared to 15.0% in the placebo arm.

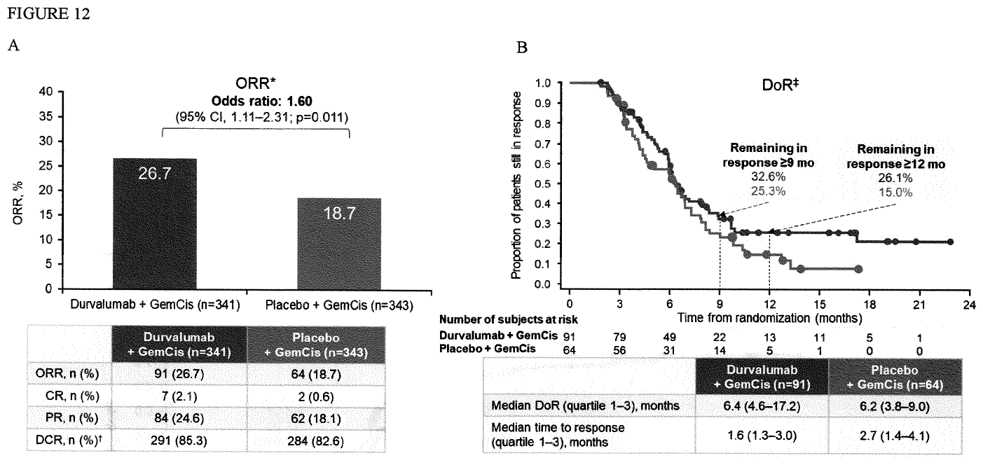

### DETAILED DESCRIPTION

The disclosure relates to methods of treating patients who have biliary tract cancer (BTC), comprising administering to the patient an anti-PD-L1 antibody in combination with chemotherapy. The disclosed methods of treatment can provide for substantial improvement in a patient's overall survival (OS), progression-free survival (PFS), objective response rate (ORR), duration of response, and/or disease control rate.

The disclosure also relates to an anti-PD-L1 antibody in combination with platinum-based chemotherapy and/or an antimetabolite-based chemotherapy for use in the treatment of biliary tract cancer. In one embodiment there is provided, an anti-PD-L1 antibody in combination with gemcitabine and/or cisplatin for use in the treatment of biliary tract cancer. In another embodiment, there is provided an anti-PD-L1 antibody in combination with platinum-based chemotherapy and/or an antimetabolite-based chemotherapy for use in the treatment of biliary tract cancer wherein treatment provides substantial improvement in a patient's overall survival (OS), progression-free survival (PFS), objective response rate (ORR), duration of response, and/or disease control rate. In another embodiment, there is provided an anti-PD-L1 antibody in combination with gemcitabine and/or cisplatin for use in the treatment of biliary tract cancer wherein treatment provides substantial improvement in a patient's overall survival (OS), progression-free survival (PFS), objective response rate (ORR), duration of response, and/or disease control rate. In a further embodiment the anti-PD-L1 is durvalumab.

The disclosure also relates to the use of an anti-PD-L1 antibody in combination with platinum-based chemotherapy and/or an antimetabolite-based chemotherapy in the manufacture of a medicament for use in the treatment of biliary tract cancer. In one embodiment, there is provided an anti-PD-L1 antibody in combination with gemcitabine and/or cisplatin for use in the treatment of biliary tract cancer. In another embodiment there is provided the use of an anti-PD-L1 antibody in combination with platinum-based chemotherapy and/or an antimetabolite-based chemotherapy in the manufacture of a medicament for use in the treatment of biliary tract cancer, wherein treatment provides substantial improvement in a patient's overall survival (OS), progression-free survival (PFS), objective response rate (ORR), duration of response, and/or disease control rate. In another embodiment there is provided the use of an anti-PD-L1 antibody in combination with gemcitabine and/or cisplatin in the manufacture of a medicament for use in the treatment of biliary tract cancer, wherein treatment provides substantial improvement in a patient's overall survival (OS), progression-free survival (PFS), objective response rate (ORR), duration of response, and/or disease control rate. In a further embodiment the anti-PD-L1 is durvalumab.

It is to be understood that the particular aspects of the disclosure are described herein are not limited to specific embodiments presented and can vary. It also will be understood that the terminology used herein is for the purpose of describing particular aspects only and, unless specifically defined herein, is not intended to be limiting. Moreover, particular embodiments disclosed herein can be combined with other embodiments disclosed herein, as would be recognized by a skilled person, without limitation.

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th ed., R. Rieger et al. (eds.), Springer Verlag (1991); and Hale & Marham, The Harper Collins Dictionary of Biology (1991). As used herein, the following terms have the meanings ascribed to them below, unless specified otherwise.

In this disclosure, “comprises,” “comprising,” “containing,” and “having,” and the like, can have the meaning ascribed to them in U.S. patent law and can mean “includes,” “including,” and the like; “consisting essentially of” or “consists essentially” likewise have the meaning ascribed in U.S. patent law and are open-ended, allowing for the presence of more than that which is recited so long as basic or novel characteristics of that which is recited are not changed by the presence of more than that which is recited, but excludes prior art aspects.

Unless specifically stated or obvious from context, the term “or,” as used herein, is understood to be inclusive. Unless specifically stated or obvious from context, the terms “a,” “an,” and “the,” as used herein, are understood to be singular or plural.

Unless specifically stated or obvious from context, the term “about,” as used herein, is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Any compositions or methods provided herein can be combined with one or more of any of the other compositions and methods provided herein.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Provided herein is a method of increasing the objective response rate in a patient with BTC, the method comprising treating the patient with an anti-PD-L1 antibody and chemotherapy.

Also provided herein is a method of extending progression-free survival in a patient with BTC, the method comprising treating the patient with an anti-PD-L1 antibody and chemotherapy.

Also provided herein is a method of treating a patient with BTC, the method comprising treating the patient with an anti-PD-L1 antibody and chemotherapy.

As used herein, “biliary tract cancers (BTC)” refers to any gastrointestinal cancer that arises from the bile ducts (cholangiocarcinoma), the gallbladder, or the ampulla of Vater (AoV).

In some embodiments, the biliary tract cancer is unresectable, locally advanced, and/or metastatic.

As used herein, the term “unresectable” refers to a cancer or tumor that cannot be removed completely through surgery. As used herein, the term “locally advanced” refers to cancer that has grown outside the body part it started in but has not yet spread to other parts of the body (Stage 3). As used herein, the term “metastatic” refers to cancer that has spread from the part of the body where it started (the primary site) to other parts of the body (Stage 4).

“Anti-PD-L1 antibody,” as used herein, refers to an antibody or antigen-binding fragment thereof that selectively binds a PD-L1 polypeptide. Exemplary anti-PD-L1 antibodies are described, for example, in U.S. Pat. Nos. 8,779,108 and 9,493,565, which are incorporated herein by reference. In some embodiments, the anti-PD-L1 antibody is durvalumab, avelumab, atezolizumab, or sugemalimab. In other embodiments, the anti-PD-L1 antibody is durvalumab, avelumab or atezolizumab. In some embodiments, the anti-PD-L1 antibody is durvalumab.

The term “durvalumab,” as used herein, refers to an antibody that selectively binds PD-L1 and blocks the binding of PD-L1 to PD-1 and CD80 receptors, as disclosed in U.S. Pat. No. 9,493,565 (wherein durvalumab is referred to as “2.14H9OPT”), which is incorporated by reference herein in its entirety. The fragment crystallizable (Fc) domain of durvalumab contains a triple mutation in the constant domain of the IgG1 heavy chain that reduces binding to the complement component C1q and the Fcγ receptors responsible for mediating antibody-dependent cell-mediated cytotoxicity (ADCC). Durvalumab can relieve PD-L1-mediated suppression of human T-cell activation in vitro and inhibits tumor growth in a xenograft model via a T-cell dependent mechanism.

As generally used herein, the terms “treat,” “treating,” “treatment,” and the like, refer to reducing, ameliorating, or slowing the progression of a disorder or disease and/or symptoms associated with a disorder or disease. It will be appreciated that, although not precluded, treating a disorder, disease, or condition does not require that the disorder, disease, or condition or associated symptoms be completely eliminated. In particular embodiments relating to BTC, “treat,” “treating,” “treatment,” can refer to achieving any one of or a combination of primary or secondary clinical endpoints.

The anti-PD-L1 antibody can be administered once or twice every three or four weeks while providing benefit to the patient. In further embodiments, the patient is administered additional follow-on doses. Follow-on doses can be administered at various time intervals depending on the patient's age, weight, clinical assessment, tumor burden, and/or other factors, including the judgment of the attending physician.

In some embodiments, multiple doses of an anti-PD-L1 antibody are administered to the patient. In some embodiments, at least three doses, at least four doses, at least five doses, at least six doses, at least seven doses, at least eight doses, at least nine doses, at least ten doses, at least fifteen doses, at least twenty-six doses, or more than at least twenty doses can be administered to the patient. In some embodiments, the anti-PD-L1 antibody is administered over a two-week period, over a three-week period, over a four-week treatment period, over a six-week treatment period, over an eight-week treatment period, over a twelve-week treatment period, over a twenty-four-week treatment period, over a one-year treatment period, or more than over a one-year treatment period.

In some embodiments, the interval between doses can be every three weeks (Q3W or q3w). In some embodiments, the interval between doses can be every four weeks (Q4W or q4w). In some embodiments, the intervals between doses can be every two months.

In some embodiments, an anti-PD-L1 antibody is administered twice over a 21-day treatment cycle (i.e., three-week period) with a first dose on day 1 of the 21-day cycle and a second dose on day 8 of the 21-day cycle. In some embodiments, the treatment cycle can be repeated 1, 2, 3, 4, 5, 6, 7, or 8 times. In some embodiments, the anti-PD-L1 antibody is administered after the completion of the final treatment cycle.

In some embodiments, the anti-PD-L1 antibody is administered after the completion of the treatment with chemotherapy.

In some embodiments, the patient is administered one or more doses of the anti-PD-L1 antibody, wherein the dose is a fixed dose of between 1000 and 2000 mg. In some embodiments, the patient is administered one or more doses of the anti-PD-L1 antibody, wherein the dose is a fixed dose of 1500 mg. In some embodiments, the patient is administered one or two doses of 1500 mg of the anti-PD-L1 antibody every three weeks or every four weeks. In some embodiments, the patient is administered one or more doses of the anti-PD-L1 antibody wherein the dose is about 20 mg/kg. In some embodiments, the patient is administered one or more doses of the anti-PD-L1 antibody, wherein the dose is about of 20 mg/kg of the anti-PD-L1 antibody every three or four weeks.

The amount of the anti-PD-L1 antibody to be administered to the patient may be adjusted, and can depend on various parameters, such as the patient's age, weight, clinical assessment, tumor burden, and/or other factors, including the judgment of the attending physician.

In some embodiments, administration of the anti-PD-L1 antibody according to the methods provided herein is through parenteral administration. For example, the anti-PD-L1 antibody can be administered by intravenous infusion or by subcutaneous injection. In some embodiments, the administration is by intravenous infusion.

The methods of treating disclosed herein further comprise chemotherapy. In some embodiments, chemotherapy comprises one or more platinum-based chemotherapeutic agents. In some embodiments, the one or more platinum-based chemotherapeutic agents is carboplatin, cisplatin, oxaliplatin, or combinations thereof. In some embodiments, chemotherapy comprises one or more antimetabolite-based chemotherapy agents. In some embodiments, the one or more antimetabolite-based chemotherapeutic agent is gemcitabine. In some embodiments, chemotherapy comprises one or more platinum-based chemotherapeutic agents in combination with one or more antimetabolite-based chemotherapy agents. In some embodiments, the chemotherapy comprises cisplatin and gemcitabine. In some embodiments, the patient is administered one or more doses of cisplatin, wherein the dose is about 25 mg/m². In some embodiments, the patient is administered one or more doses of gemcitabine, wherein the dose is about 1000 mg/m².

In some embodiments, the anti-PD-L1 antibody is administered concurrently with chemotherapy. In some embodiments, the anti-PD-L1 antibody and chemotherapy are administered within about three days of each other. In some embodiments, the anti-PD-L1 antibody and chemotherapy are administered within about two days of each other. In some embodiments, the anti-PD-L1 antibody and chemotherapy are administered within about one day of each other. In some embodiments, the anti-PD-L1 antibody and chemotherapy are administered concurrently (for example by simultaneous (same day) administration). In some embodiments, the anti-PD-L1 antibody is administered on day 1 of the chemotherapy treatment cycle. In some embodiments, a first dose of the anti-PD-L1 antibody and the chemotherapy are administered on day 1 of a 21-day treatment cycle, and a second dose of the anti-PD-L1 antibody is administered on day 8 of a 21-day treatment cycle.

In some embodiments, the observed toxicities of the treatment with an anti-PD-L1 antibody and chemotherapy combination are similar to those commonly seen with either chemotherapy or immunotherapy alone.

In some embodiments, the patient has been previously treated for biliary tract cancer with surgery and/or an adjuvant therapy and/or chemotherapy alone. In some embodiments, the patient has not been previously treated for biliary tract cancer. In some embodiments, the patient has previously been treated for biliary tract cancer and the treatment was not curative. In some embodiments, the patient has previously been treated for biliary tract cancer and the treatment was curative but the patient has developed recurrent disease. In some embodiments, the patient has developed recurrent disease more than 2, 3, 4, 5, 6, 7, 8, 9, or more than 9 months after surgery with curative intent. In some embodiments, the patient has developed recurrent disease more than 2, 3, 4, 5, 6, 7, 8, 9, or more than 9 months after the completion of adjuvant therapy.

In some embodiments, the methods of treatment disclosed herein provide an increase in PFS relative to placebo. In some embodiments, the methods provide an increase in ORR relative to placebo. In some embodiments, the methods provide an increase in OS versus placebo.

Overall Survival (OS) relates to the time period beginning on the date of treatment until death due to any cause. OS may refer to overall survival within a period of time such as, for example, 12 months, 18 months, 24 months, and the like. Such periods of time can be identified, for example, as “OS24” which refers to the number (%) of patients who are alive at 24 months after treatment onset per the Kaplan-Meier estimate of overall survival at 24 months.

Progression-Free Survival (PFS) relates to the time period beginning on the date of treatment until the date of objective disease progression (RECIST 1.1) or death (by any cause in the absence of progression). In some embodiments, the methods of the disclosure provide for increase in PFS. In some embodiments, the methods provide for PFS of at least 9 months to at least about 24 months (e.g., at least 9, 10, 11, 12, 13, 14, 15, 16, 17, 18, 19, 20, 21, 22, 23, 24, or more than 24 months, and up to about 5 years).

Objective Response Rate (ORR) refers to the number (%) of patients with at least one visit response of Complete Response (CR) or partial response (PR) per RECIST 1.1.

Duration of Response (DoR) refers to the time from the date of first documented response until the first date of documented progression or death in the absence of disease progression (i.e., date of PFS event or censoring−date of first response+1).

Disease Control Rate (DCR) refers to the rate of best objective response of CR, PR, or stable disease (SD) according to RECIST 1.1.

Patient Reported Outcomes (PRO) is a type of clinical outcome assessment. PROs are used to evaluate the effectiveness of treatments in cancer clinical studies. PRO-related assessments are evaluated using quality-of-life questionnaires. PROs use direct patient input which identify significant symptomatic, functional, and health related quality of life (HRQOL) burdens experienced by patients with advanced BTC. The PRO endpoints are measured using the European Organisation for Research and Treatment of Cancer (EORTC) 30-Item Core Quality of Life Questionnaire (QLQ-C30) and the complementary EORTC-21-Item Cholangiocarcinoma and Gallbladder Cancer Quality of Life Questionnaire (QLQ-BIL21), the Patient-Reported Outcomes-Common Terminology Criteria for Adverse Events (PRO CTCAE), Patient Global Impression of Severity (PGIS), and EuroQOL 5-dimension, 5 level health state utility index (EQ-5D-5L). These questionnaires show the overall influence of the benefits and toxicity of the treatment from a patient's perspective and aid in understanding of the benefit/risk evaluation. The EORTC QLQ-C30 and the EORTC QLQ-BIL21 assess the impact of treatment and disease state on symptoms, impacts, and HRQOL. The EORTC scales include many of the key BTC symptoms and impacts, such as abdominal pain, fatigue, pruritus, jaundice, lack of appetite, physical functioning, and insomnia.

The EORTC QLQ-C30 includes 30 items and assesses HRQOL/health status through the following 9 multi item scales: 5 functional scales (i.e., physical, role, cognitive, emotional, and social), 3 symptom scales (i.e., fatigue, pain, and nausea and vomiting), and 1 global health status/QoL scale. It also includes 6 single-item symptom/impact measures: dyspnea, insomnia, appetite loss, constipation, diarrhea, and financial difficulties.

The EORTC QLQ-BIL21 is used as an accompaniment to the EORTC QLQ-C30. The EORTC QLQ-BIL21 consists of 21 questions: 3 single-item assessments relating to treatment side effects, difficulties with drainage bags/tubes, and concerns regarding weight loss, in addition to 18 items grouped into 5 scales, which are pain symptoms (4 items), tiredness symptoms (3 items), jaundice symptoms (3 items), anxiety symptoms (4 items), and eating symptoms (4 items).

PROs of the Patient-Reported Outcomes-Common Terminology Criteria for Adverse Events (PRO CTCAE), Patient Global Impression of Severity (PGIS), and EuroQoL 5-dimension, 5 level health state utility index (EQ-5D-5L) assess treatment benefit and risk from the patient's perspective.

The PRO-CTCAE assesses tolerability from the patients' perspective. The PRO-CTCAE is an item-bank of symptoms. Six different symptomatic adverse events (AEs) are included for the PRO-CTCAE.

Patient Global Impression of Severity (PGIS) assess how a patient perceives his/her overall severity of cancer symptoms over the past 7 days. This is a single-item questionnaire, and patients will choose from response options ranging from “no symptoms” to “very severe.”

EQ-5D-5L is a questionnaire that provides a simple descriptive profile of health and a single index value for health status for economic appraisal (EuroQol Group, 2015). The EQ-5D-5L questionnaire comprises 5 questions that cover 5 dimensions of health (i.e., mobility, self-care, usual activities, pain/discomfort, and anxiety/depression). Respondents also assess their health today using the EQ-visual analog scale (VAS), which ranges from 0 (worst imaginable health) to 100 (best imaginable health).

### EXAMPLES

### Example 1: Efficacy of Durvalumab in Combination with Chemotherapy for Patients with First-Line Advanced Biliary Tract Cancers (TOPAZ-1)

### Overview

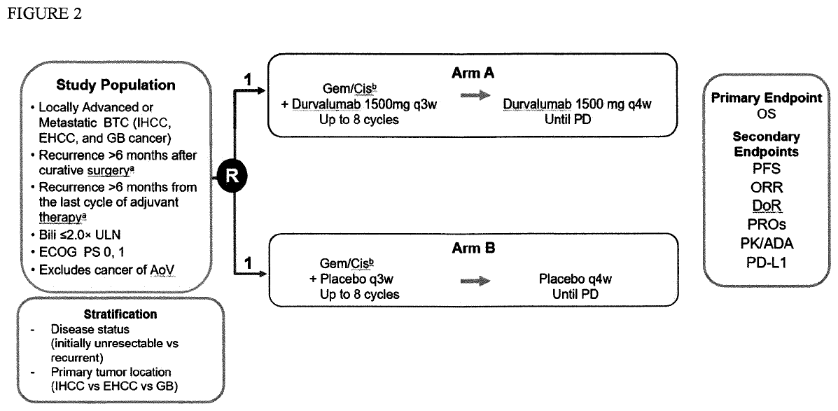

This is a randomized, double-blind, placebo controlled, multi-center, global, Phase III study which determined the efficacy and safety of first-line treatment with durvalumab in combination with gemcitabine/cisplatin versus placebo in combination with gemcitabine/cisplatin in patients with previously untreated, unresectable, locally advanced, or metastatic BTC. An overview of the study design is shown in FIG. 2.

Approximately 672 patients with previously untreated, unresectable, locally advanced, or metastatic BTC will be randomized 1:1 to receive one of the following treatments (approximately 336 patients are to be randomized to each arm):

Arm A: Durvalumab plus gemcitabine/cisplatin combination therapy. Durvalumab 1500 mg via intravenous (IV) infusion q3w, starting on Cycle 1 in combination with cisplatin 25 mg/m² and gemcitabine 1000 mg/m² (each administered on Days 1 and 8 q3w) up to 8 cycles, followed by durvalumab 1500 mg as monotherapy q4w until clinical progression or RECIST 1.1-defined radiological progressive disease (PD), unless there is unacceptable toxicity, withdrawal of consent, or another discontinuation criterion is met.

Arm B: Placebo plus gemcitabine/cisplatin therapy. Placebo via IV infusion q3w, starting on Cycle 1 in combination with cisplatin 25 mg/m² and gemcitabine 1000 mg/m² (each administered on Days 1 and 8 q3w) up to 8 cycles, followed by placebo monotherapy q4w until clinical progression or RECIST 1.1-defined radiological PD, unless there is unacceptable toxicity, withdrawal of consent, or another discontinuation criterion is met.

The primary objective of the study is to confirm the superiority of Arm A compared to Arm B in terms of overall survival (OS) in patients with first-line advanced BTC.

Randomization will be stratified by disease status (initially unresectable versus recurrent) and primary tumor site (intrahepatic cholangiocarcinoma versus extrahepatic cholangiocarcinoma versus gallbladder cancer).

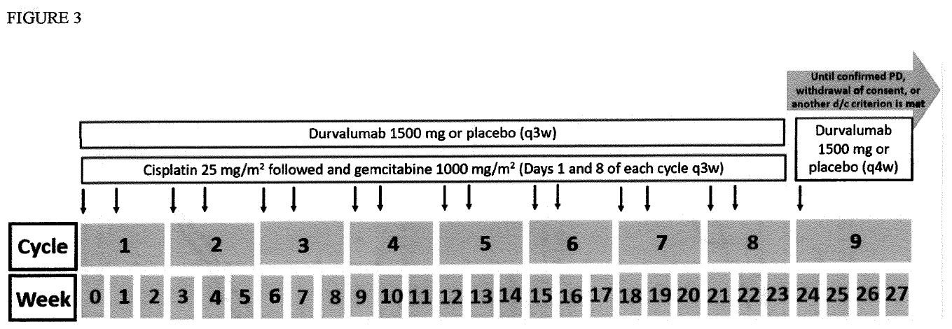

Patients will receive cisplatin 25 mg/m² and gemcitabine 1000 mg/m² (each administered on Days 1 and 8 q3w) plus durvalumab 1500 mg (Arm A) or placebo (Arm B) via IV infusion q3w, starting on Cycle 1, for up to 8 cycles. After treatment with gemcitabine/cisplatin is complete, the patients will receive 1500 mg durvalumab (Arm A) or placebo (Arm B) via IV infusion q4w until clinical progression or RECIST 1.1-defined radiological PD, unless there is unacceptable toxicity, withdrawal of consent, or another discontinuation criterion is met (FIG. 3). (Note that if a patient's weight falls to ≤30 kg, the patient should receive a weight-based dosing equivalent to 20 mg/kg of durvalumab [or placebo] q3w or q4w after consultation between the Investigator and the Study Physician, until the weight improves to >30 kg, at which point the patient should start receiving the fixed dosing of durvalumab 1500 mg [or placebo] q3w or q4w.)

### Objectives and Endpoints

The primary endpoint of the proposed study will be overall survival (OS), defined as the time from the date of randomization until death due to any cause. OS is considered the most reliable cancer endpoint in this Phase III study supported by the U.S. Food and Drug Administration and European Medicines Agency guidelines (U.S. Food and Drug Administration, 2007; European Medicines Agency, 2012). OS is considered the most appropriate endpoint, given that the median overall survival (mOS) in patients with advanced BTC receiving gemcitabine and cisplatin is <1 year (Valle et al., 2010; Okusaka et al., 2010).

The secondary efficacy endpoints will include progression free survival (PFS), objective response rate (ORR), duration of response (DoR), disease control rate (DCR), and patient reported outcome (PRO) measures. All tumor-related endpoints will be assessed by the site Investigator according to RECIST 1.1; ORR and DoR will also be assessed by a BICR according to RECIST 1.1 for IA-1.

The objectives and endpoints of the study are outlined in Table 1.

TABLE 1
Objectives and Endpoints

| Objectives | Endpoints |
|---|---|
| Primary objective | Endpoint/Variable |
| To assess the efficacy of Arm A compared to Arm B | OS |
| in terms of OS in patients with advanced BTC | |
| Objectives | Endpoints |
| Secondary objectives | Endpoints/Variables |
| To further assess the efficacy of Arm A compared to | PFS, ORR, and DoR according to RECIST 1.1 using |
| Arm B in terms of PFS, ORR, and DoR in patients | Investigator assessments |
| with advanced BTC | |
| For IA-1: To summarize the efficacy of Arm A | ORR and DoR according to RECIST 1.1 using BICR |
| compared to Arm B in terms of ORR and DoR in | assessments |
| patients with advanced BTC | |
| To assess disease-related symptoms, impacts, and | EORTC QLQ-C30: Global health status/QoL and |
| HRQoL in patients treated with Arm A compared to | impacts (e.g., physical function); multi-term symptoms |
| Arm B | (e.g., fatigue); and single items (e.g., appetite loss, |
| | insomnia) |
| | EORTC QLQ-BIL21: Single-item symptoms (e.g., |
| | abdominal pain [item 42], pruritus [item 36], jaundice |
| | [item 35]) |
| To assess the efficacy of Arm A compared to Arm B | Association of PD-L 1 expression level with PFS, ORR, |
| by PD-L1 expression | DoR, and DCR according to RECIST 1.1 using |
| | Investigator assessments and OS |
| To assess the PK of durvalumab when used in | Serum concentration of durvalumab (peak and trough |
| combination with gemcitabine/cisplatin | concentrations) |
| To investigate the immunogenicity of durvalumab | Reporting tiered results of ADAs for durvalumab |
| Safety objective | Endpoint/Variable |
| To assess the safety and tolerability profile of Arm A | AEs, physical examinations, laboratory findings, WHO |
| compared to Arm B in patients with advanced BTC | ECOG/PS, ECG and vital signs |
| Exploratory objectives | Endpoints/Variables |
| To investigate the efficacy of Arm A compared to | Association of candidate biomarkers including but not |
| Arm B by candidate biomarkers (for example but not | limited to TMB, MSI and/or tumor mutations with: OS |
| limited to TMB and MSI) that may correlate with | and PFS, ORR, DoR, and DCR according to |
| drug activity or identify patients likely to respond to | RECIST 1.1 using Investigator assessments |
| treatment | |
| To evaluate circulatory-based biomarkers and | Association with circulatory-based biomarkers, |
| associations with efficacy parameters, including, but | including, but not limited to, ctDNA-based TMB, whole |
| not limited to, ctDNA | blood gene expression, etc., with efficacy assessments |
| It is not applicable in China. | |
| To assess patient-reported treatment tolerability using | PRO-CTCAE (pre-selected items based on treatment |
| PRO-CTCAE and global assessment of treatment | arms) and global assessment of treatment tolerability |
| tolerability | (QLQ-BIL21 item 49) |
| To assess the patients' global impression of the | PGIS |
| severity of cancer symptoms | |
| To explore the impact of treatment and disease state | The EQ-5D-5L health state utility instrument will be |
| on health state utility using the EQ-5D-5L | used to derive health state utility based on |
| | patient-reported data. |
| Objectives | Endpoints |
| To explore the impact of treatment and disease on | The HOSPAD module will be used to collect |
| healthcare resource use | information on key healthcare resource use beyond study |
| | mandated visits. |

Note:
Patients in Arm A will receive durvalumab plus gemcitabine/cisplatin combination therapy; patients in Arm B will receive placebo plus gemcitabine/cisplatin therapy.
ADA Antidrug antibody; AE Adverse event; BICR Blinded independent central review; BTC Biliary tract cancer; ctDNA Circulating tumor deoxyribonucleic acid; DCR Disease control rate; DoR Duration of response; ECOG Eastern Cooperative Oncology Group; EORTC European Organisation for Research and Treatment of Cancer; EQ-5D-5L EuroQoL 5-dimension, 5-level health state utility index; HOSPAD Hospital resource use module; HRQoL Health-related quality of life; IA-1 Interim Analysis 1; MSI Microsatellite instability; ORR Objective response rate; OS Overall survival; PD-L1 Programmed cell death ligand-1; PFS Progression-free survival; PGIS Patient Global Impression of Severity; PK Pharmacokinetics; PRO-CTCAE Patient-Reported Outcomes-Common Terminology Criteria for Adverse Events; PS Performance status; QLQ-BIL21 21-Item Cholangiocarcinoma and Gallbladder Cancer Quality of Life Questionnaire; QLQ-C30 30-Item Core Quality of Life Questionnaire; QoL Quality of life; RECIST Response Evaluation Criteria in Solid Tumors; TMB Tumor mutational burden; WHO World Health Organization.

### Study Population

The study population includes patients ≥18 years of age with previously untreated, unresectable, locally advanced, or metastatic BTC (e.g., intrahepatic or extrahepatic cholangiocarcinoma, gallbladder cancer). Cancer of AoV has a different genetic profile than other subtypes of BTC (Yachida et al., 2016). To minimize the diversity of the study population, the patients with cancer of AoV were excluded from the study.

Each patient should meet all of the inclusion criteria and none of the exclusion criteria for this study in order to be randomized to a study intervention.

In this protocol, “enrolled” patients are defined as those who sign an informed consent and receive an enrollment number. “Randomized” patients are defined as those who undergo randomization and receive a randomization number.

Patients who do not meet the entry requirements are screen failures. Prospective approval of protocol deviations to recruitment and enrollment criteria, also known as protocol waivers or exemptions, is not permitted.

### Inclusion Criteria

Patients are eligible to be included in the study only if all of the following inclusion criteria and none of the exclusion criteria apply:

### Informed Consent:

1. Capable of giving signed informed consent, which includes compliance with the requirements and restrictions listed in the informed consent form (ICF) and in this protocol.
2. Provision of a signed and dated written ICF prior to any mandatory study-specific procedures, sampling, and analyses.
3. Provision of a signed and dated written informed consent prior to the collection of sample for optional genetic analysis.

### Age

4. Age ≥18 years at the time of screening. For patients aged <20 years and enrolled in Japan, a written informed consent should be obtained from the patient and his/her legally acceptable representative.

### Type of Patient and Disease Characteristics

5. Histologically confirmed, unresectable, advanced, or metastatic adenocarcinoma of biliary tract, including cholangiocarcinoma (intrahepatic or extrahepatic) and gallbladder carcinoma.
6. Patients with previously untreated disease if unresectable or metastatic at initial diagnosis will be eligible.
7. Patients who developed recurrent disease >6 months after surgery with curative intent and, if given, >6 months after the completion of adjuvant therapy (chemotherapy and/or radiation) will be eligible.
8. A World Health Organization (WHO)/ECOG PS of 0 or 1 at enrollment.
9. At least 1 lesion that qualifies as a RECIST 1.1 Target Lesion (TL) at baseline.
10. No prior exposure to immune-mediated therapy, including, but not limited to, other anti CTLA 4, anti PD-1, anti-PD-L1, and anti-PD-L2 antibodies, excluding therapeutic anticancer vaccines.
11. Adequate organ and marrow function, as defined below:
Hemoglobin ≥9.0 g/dL.
Absolute neutrophil count ≥1.5×109/L.
Platelet count ≥100×109/L.
Serum bilirubin≤2.0×the upper limit of normal (ULN); This will not apply to patients with confirmed Gilbert's syndrome. Any clinically significant biliary obstruction should be resolved before randomization.
Alanine aminotransferase (ALT) and aspartate aminotransferase (AST)≤2.5×ULN; for patients with hepatic metastases, ALT and AST≤5×ULN.
Creatinine clearance (CL)>50 mL/min per 24 hr urine or as calculated by Cockcroft-Gault (using actual body weight):

### Males.

Creatinine CL(mL/min)=(Weight(kg)×(140−Age))/(72×serum creatinine(mg/dL)).

### Females:

Creatinine CL(mL/min)=(Weight(kg)×(140−Age)×0.85)/(72×serum creatinine(mg/dL))
12. Patients must have a life expectancy of at least 12 weeks at the time of screening.

### Weight

13. Body weight >30 kg.

### Sex

14. Male and/or female.

### Other

Patients must provide a recent tumor biopsy or an available unstained archived tumor tissue sample in a quantity sufficient to allow for analysis (taken≤3 years prior to screening). The tumor lesions to be used for biopsy should not be those used as RECIST TLs, unless there are no other lesions suitable for biopsy.

Patients with HBV infection (as characterized by positive hepatitis B surface antigen [HBsAg] and/or anti-hepatitis B core antibodies (anti-HBc) with detectable HBV deoxyribonucleic acid (DNA) [≥10 IU/mL or above the limit of detection per local laboratory]) must receive antiviral therapy prior to randomization per institutional practice to ensure adequate viral suppression. Patients must remain on antiviral therapy for the study duration and for 6 months after the last dose of study treatment. Patients who test positive for anti-HBc with undetectable HBV DNA (<10 IU/mL or under the limit of detection per local laboratory) do not require antiviral therapy unless HBV DNA exceeds 10 IU/mL or reaches detectable limits per local laboratory during the course of treatment. Patients with active co-infection of HBV and HCV as evidenced by positive anti-HCV antibody and actively co-infected with HBV and hepatitis D virus are not eligible.

### Exclusion Criteria

### Medical Conditions:

Ampullary carcinoma.

History of allogeneic organ transplantation.

Active or prior documented autoimmune or inflammatory disorders (including inflammatory bowel disease (e.g., colitis or Crohn's disease), diverticulitis (with the exception of diverticulosis), systemic lupus erythematosus, Sarcoidosis syndrome, or Wegener syndrome (granulomatosis with polyangiitis, Graves' disease, rheumatoid arthritis, hypophysitis, uveitis, etc.). The following are exceptions to this criterion:

Patients with vitiligo or alopecia.

Patients with hypothyroidism (e.g., following Hashimoto syndrome) stable on hormone replacement.

Any chronic skin condition that does not require systemic therapy.

Patients without an active disease in the last 5 years may be included but only after consultation with the Study Physician.

Patients with celiac disease controlled by diet alone.

Uncontrolled intercurrent illness, including, but not limited to, ongoing or active infection, symptomatic congestive heart failure, uncontrolled hypertension, unstable angina pectoris, uncontrolled cardiac arrhythmia, active interstitial lung disease (ILD), serious chronic gastrointestinal conditions associated with diarrhea, or psychiatric illness/social situations that would limit compliance with study requirement, substantially increase the risk of incurring AEs, or compromise the ability of the patient to give written informed consent.

History of another primary malignancy, except for:

Malignancy treated with curative intent and with no known active disease ≥5 years before the first dose of investigational product (IP) and of low potential risk for recurrence.

Adequately treated non-melanoma skin cancer or lentigo maligna without evidence of disease.

Adequately treated carcinoma in situ without evidence of disease.

History of leptomeningeal carcinomatosis.

History of active primary immunodeficiency.

Active infection, including tuberculosis (clinical evaluation that includes clinical history, physical examination and radiographic findings, and tuberculosis testing in line with local practice), or human immunodeficiency virus (positive HIV 1/2 antibodies).

Any unresolved toxicity NCI Common Terminology Criteria for Adverse Event (CTCAE) Grade ≥2 from a previous anticancer therapy, with the exception of alopecia, vitiligo, and the laboratory values defined in the inclusion criteria.

Patients with Grade ≥2 neuropathy will be evaluated on a case-by-case basis after consultation with the Study Physician.

Patients with irreversible toxicity, not reasonably expected to be exacerbated by treatment with durvalumab, may be included only after consultation with the Study Physician.

Brain metastases or spinal cord compression (including asymptomatic and adequately treated disease). Patients with suspected brain metastases at screening should have an MRI (preferred) or CT scan, each preferably with IV contrast, of the brain prior to study entry.

Known allergy or hypersensitivity to any of the study drugs or any of the study drug excipients.

### Prior/Concomitant Therapy

Any concurrent chemotherapy, IP, biologic, or hormonal therapy for cancer treatment. Concurrent use of hormonal therapy for non-cancer-related conditions (e.g., hormone replacement therapy) is acceptable.

Radiation therapy, including palliative radiation, is not allowed before the study, with an exception of radiation given in an adjuvant setting.

Receipt of live attenuated vaccine within 30 days prior to the first dose of IP. Note that patients, if enrolled, should not receive live vaccine while receiving IP and up to 30 days after the last dose of IP.

Major surgical procedure (as defined by the Investigator) within 28 days prior to the first dose of IP. Note that minor surgery of isolated lesions for palliative intent is acceptable if performed more than 14 days prior to the first dose of IP.

Patients who have received prior immune-mediated therapy, including, but not limited to, other anti-PD-1, anti PD-L1, or anti CTLA-4.

Prior locoregional therapy such as radioembolization.

Current or prior use of immunosuppressive medication within 14 days before the first dose of durvalumab. The following are exceptions to this criterion:

Intranasal, inhaled, or topical steroids or local steroid injections (e.g., intra-articular injection).

Systemic corticosteroids at physiologic doses not to exceed 10 mg/day of prednisone or its equivalent.

Steroids as premedication for hypersensitivity reactions (e.g., CT scan premedication).

### Prior/Concurrent Clinical Study Experience

Participation in another clinical study with an IP administered in the last 3 months.

Previous IP assignment in the present study.

Concurrent enrollment in another clinical study, unless it is an observational (non-interventional) clinical study or during the follow-up period of an interventional study.

Prior randomization or treatment in a previous durvalumab clinical study, regardless of treatment arm assignment.

### Other Exclusions

Female patients who are pregnant or breastfeeding or male or female patients of reproductive potential who are not willing to use effective birth control from screening to 180 days after the last dose of gemcitabine/cisplatin or 90 days after the last dose of durvalumab/placebo monotherapy.

Judgment by the Investigator that the patient should not participate in the study if the patient is unlikely to comply with study procedures, restrictions, and requirements.

### Lifestyle Restrictions

The following restrictions apply while the patient is receiving IP and for the specified times before and after:

Female patient of child-bearing potential.

Female patients of childbearing potential who are not abstinent and intend to be sexually active with a non-sterilized male partner must use at least 1 highly effective method of contraception from the time of screening throughout the total duration of the drug treatment and the drug washout period (180 days after the last dose of gemcitabine/cisplatin or 90 days after the last dose of durvalumab/placebo monotherapy). Non-sterilized male partners of female patients of childbearing potential must use a male condom plus spermicide throughout this period. Cessation of birth control after this point should be discussed with a responsible physician. True abstinence is acceptable when this is in line with the preferred and usual lifestyle of the patient. Periodic abstinence, the rhythm method, and the withdrawal method are not acceptable methods of contraception. Female patients should refrain from breastfeeding throughout this period.

Male patients with a female partner of childbearing potential.

Non-sterilized male patients who are not abstinent and intend to be sexually active with a female partner of childbearing potential must use a male condom plus spermicide from the time of screening throughout the total duration of the drug treatment and the drug washout period (180 days after the last dose of gemcitabine/cisplatin or 90 days after the last dose of durvalumab/placebo monotherapy). True abstinence is acceptable when this is in line with the preferred and usual lifestyle of the patient. Periodic abstinence, the rhythm method, and the withdrawal method are not acceptable methods of contraception. Male patients should refrain from sperm donation throughout this period.

Female partners (of childbearing potential) of male patients must also use a highly effective method of contraception throughout this period.

Note that females of childbearing potential are defined as those who are not surgically sterile (i.e., bilateral salpingectomy, bilateral oophorectomy, or complete hysterectomy) or post-menopausal.

Women will be considered post-menopausal if they have been amenorrheic for 12 months without an alternative medical cause. The following age-specific requirements apply:

Women <50 years of age would be considered post-menopausal if they have been amenorrheic for ≥12 months following cessation of exogenous hormonal treatments and if they have luteinizing hormone and follicle-stimulating hormone levels in the post-menopausal range for the institution.

Women >50 years of age would be considered post-menopausal if they have been amenorrheic for ≥12 months following cessation of all exogenous hormonal treatments, had radiation-induced menopause with the last menses ≥1 year ago, or had chemotherapy-induced menopause with the last menses ≥1 year ago.

Women who are surgically sterile (i.e., bilateral salpingectomy, bilateral oophorectomy, or complete hysterectomy) are eligible.

Highly effective methods of contraception, defined as one that results in a low failure rate (i.e., <1% per year) when used consistently and correctly. Note that some contraception methods are not considered highly effective (e.g., male or female condom with or without spermicide; female cap, diaphragm, or sponge with or without spermicide; non-copper-containing intrauterine device; progestogen-only oral hormonal contraceptive pills where inhibition of ovulation is not the primary mode of action [excluding Cerazette/desogestrel, which is considered highly effective]; and triphasic combined oral contraceptive pills).

Serum levels of contraceptive hormones may be altered by concomitant use of other drugs. Therefore, drug interactions should always be considered when prescribing hormonal contraception; there could be a risk of contraceptive failure or other adverse effects.

For patients receiving gemcitabine/cisplatin (both arms), the local prescribing information relating to contraception, the time limits for such precautions, and any additional restrictions for these agents should be followed.

### Concomitant Therapy

The Investigator must be informed as soon as possible about any medication taken from the time of screening until the end of the clinical treatment phase of the study, including the 90-day follow-up period following the last dose of study drug.

Any medication or vaccine, including over-the-counter or prescription medicines, vitamins, and/or herbal supplements that the patient is receiving at the time of enrollment or receives during the study, must be recorded along with:

### Reason for Use:

### Dates of Administration, Including the Start and End Dates

### Dosage Information, Including the Dose, Unit, and Frequency

Patients must be instructed not to take any medications, including over-the-counter products, without first consulting with the Investigator.

Restricted, prohibited, and permitted concomitant medications are described in the following tables. Refer also to the Dosing Modification and Toxicity Management Guidelines. For gemcitabine/cisplatin, refer to the local prescribing information with regard to warnings, precautions, and contraindications.

TABLE 2
Prohibited concomitant medications

| Prohibited medication/class of drug | Usage |
|---|---|
| Any investigational anticancer therapy | Should not be given concomitantly while the |
| other than those under investigation in this | patient is on study treatment. |
| study | |
| mAbs against CTLA-4, PD-1, or PD-L1 | Should not be given concomitantly while the |
| other than those under investigation in this | patient is on study treatment. |
| study | |
| Any concurrent chemotherapy, | Should not be given concomitantly while the |
| radiotherapy, immunotherapy, or biologic | patient is on study treatment. (Concurrent use of |
| or hormonal therapy for cancer treatment | hormones for non-cancer-related conditions |
| other than those under investigation in this | [e.g., insulin for diabetes and hormone |
| study | replacement therapy] is acceptable. Local |
| | treatment of isolated lesions, excluding TLs, for |
| | palliative intent is acceptable [e.g., radiotherapy |
| | for bone metastasis].) |
| Live attenuated vaccines | Should not be given through 30 days after the |
| | last dose of IP. |
| Immunosuppressive medications, | Should not be given concomitantly or used for |
| including, but not limited to, systemic | premedication prior to the IO infusions. The |
| corticosteroids at doses exceeding 10 | following are allowed exceptions: |
| mg/day of prednisone or equivalent, | Use of immunosuppressive medications |
| methotrexate, azathioprine, and tumor | for the management of IP-related AEs |
| necrosis factor-α blockers | Short-term premedication for patients |
| | receiving gemcitabine/cisplatin, where |
| | the prescribing information for the agent |
| | requires the use of steroids for |
| | documented hypersensitivity reactions |
| | Use in patients with contrast allergies |
| | In addition, use of inhaled, topical, and |
| | intranasal corticosteroids is permitted |
| | A temporary period of steroids, which |
| | will be allowed if clinically indicated and |
| | considered to be essential for the |
| | management of |
| | non-immunotherapy-related events |
| | experienced by the patient (e.g., chronic |
| | obstructive pulmonary disease, radiation, |
| | nausea) |
| EGFR TKIs | Should not be given concomitantly. |
| | Should be used with caution in the 90 days after |
| | the last dose of durvalumab. |
| | Increased incidences of pneumonitis (with third- |
| | generation EGFR TKIs) and transaminase |
| | increases (with first-generation EGFR TKIs) |
| | have been reported when durvalumab has been |
| | given concomitantly. |
| Herbal and natural remedies which may | Should not be given concomitantly, unless |
| have immune-modulating effects | agreed by the Sponsor. |

TABLE 3
Supportive medications

| Supportive medication/class of drug | Usage |
|---|---|
| Concomitant medications or treatments | To be administered as |
| (e.g., acetaminophen or diphenhydramine) | prescribed by the Investigator |
| deemed necessary to provide adequate | |
| prophylactic or supportive care, except for | |
| medications identified as “prohibited,” as | |
| listed above | |
| Best supportive care (including antibiotics, | Should be used, when |
| nutritional support, correction of metabolic | necessary, for all patients |
| disorders, optimal symptom control, and | |
| pain management [including palliative | |
| radiotherapy to NTLs [i.e., for bone | |
| metastasis], etc.]) | |
| Inactivated viruses, such as those in the | Permitted |
| influenza vaccine | |

NTL Non-Target Lesion.

### Other Concomitant Treatment

Medication other than that described above, which is considered necessary for the patient's safety and well-being, may be given at the discretion of the Investigator and recorded in the appropriate sections of the Case Report Form (CRF).

### Rescue Medication

As a result of immune-mediated adverse events (imAEs) that could potentially be experienced by patients on durvalumab, steroids and other immunosuppressant rescue medications have to be made available to this patient population. The 2 products that fall into the category of immunosuppressants are infliximab (e.g., for colitis) and mycophenolate (e.g., for hepatitis). These rescue medications must be receipted, controlled, and dispensed by the unblinded pharmacist and stored according to the labelled storage conditions, with temperature excursions reported accordingly by the unblinded pharmacist. If required for use as a result of an imAE, then the IVRS/IWRS will provide to the unblinded pharmacists the kit identification number to be allocated to the patient at the time. Blinded and unblinded access and notifications will be controlled using the IVRS/IWRS.

### Dose Modification

Dose delays are permitted for durvalumab (or placebo) therapy. However, dose reduction for durvalumab (or placebo) therapy is not permitted.

The Investigator should follow local standard clinical practice regarding dose modifications for gemcitabine/cisplatin.

With regard to gemcitabine/cisplatin, patients may delay and subsequently resume dosing per local standard clinical practice. If dosing must be delayed for reasons other than treatment-related toxicity, dosing will occur as soon as feasible. In the event that chemotherapy is discontinued due to treatment-related toxicity, treatment with durvalumab/placebo may continue at the Investigator's discretion when toxicity resolves to Grade 2 or less; in that case, durvalumab/placebo monotherapy will be administered q4w.

During the first 8 cycles of durvalumab or placebo in combination with gemcitabine/cisplatin, if durvalumab or placebo must be discontinued due to durvalumab- or placebo-related toxicity, the patient must complete the treatment discontinuation visit, and enter the Follow-Up Period. Treatment with Gemcitabine/cisplatin may continue at the discretion of the investigator during the follow-up period and should reported in the eCRF as subsequent anticancer treatment. Subsequent anticancer treatment should be held until the toxicity resolves to Grade 2 or less.

### Discontinuation of Treatment and Subject Withdrawal

### Discontinuation of Study Treatment

An individual patient will not receive any further study treatment (durvalumab, placebo, or gemcitabine/cisplatin) if any of the following occur in the patient in question:

Withdrawal of consent from further treatment with IP. The patient is, at any time, free to discontinue treatment, without prejudice to further treatment. A patient who discontinues treatment is normally expected to continue to participate in the study (e.g., for safety and survival follow-up), unless he/she specifically withdraws consent to all further participation in any study procedures and assessments.

Any AE that meets the criteria for discontinuation as defined in the Dosing Modification and Toxicity Management Guidelines or as defined in the local prescribing information for gemcitabine/cisplatin. NOTE: Patients who permanently discontinue treatment with durvalumab or placebo for durvalumab/placebo related toxicity, should complete the treatment discontinuation visit and enter the Follow-up Period. If this occurs while patient is receiving SOC (e.g., cycles 1-8), treatment with gemcitabine/cisplatin may continue during the follow up period at the investigator's discretion as described in herein.

Pregnancy or intent to become pregnant.

Non-compliance with the study protocol that, in the opinion of the Investigator, warrants withdrawal from treatment with IP (e.g., refusal to adhere to scheduled visits).

Initiation of alternative anticancer therapy, including another investigational agent.

### Efficacy Assessments

This study will evaluate the primary endpoint of OS. The secondary efficacy endpoints of PFS, ORR, DoR, and DCR will be derived using Investigator RECIST 1.1 assessments; ORR and DoR will also be assessed by a BICR according to RECIST 1.1 for IA-1. PRO-related assessment will be evaluated using quality-of-life questionnaires.

Baseline RECIST 1.1 assessments (selection of TLs and Non-Target Lesions) should be performed on images from CT scans (preferred) or MRI scans, each preferably with IV contrast, of the chest, abdomen (including the entire liver and both adrenal glands), and pelvis, acquired no more than 28 days before the date of randomization and, ideally, should be performed as close as possible to and prior to the date of randomization. Additional anatomy should be imaged based on the signs and symptoms of individual patients at baseline and follow-up. It is important to follow the tumor assessment schedule as closely as possible. If an unscheduled assessment is performed (e.g., to investigate clinical signs/symptoms of progression), every attempt should be made to perform the subsequent assessments at their next regularly scheduled visit. The follow-up scan collected after a RECIST 1.1-defined radiological PD should be performed preferably at the next (and no later than the next) scheduled imaging visit and no less than 4 weeks after the prior assessment of PD; this follow-up scan is evaluated using the criteria outlined in Appendix A.

### Survival Assessments

Assessments for survival must be made every 2 months following treatment discontinuation for those patients who have not yet entered long term follow up (i.e., all China cohort patients). Patients who discontinued treatment and are in long term follow up (after original FA DCO) should continue survival assessments every 3 months during the long-term follow up. Survival information may be obtained via telephone contact with the patient or the patient's family or by contact with the patient's current physician. Details of the first and subsequent therapies for cancer, after discontinuation of treatment, will be collected.

In addition, patients on treatment or in survival follow-up will be contacted following the DCO for the interims, final (i.e., IA-1, IA-2, and FA) and the additional exploratory long-term OS analyses to provide complete survival data. These contacts should generally occur within 7 days of the DCO.

### Clinical Outcome Assessments

A clinical outcome assessment is an assessment of a clinical outcome made through report by a clinician, a patient, or a non-clinician observer or through a performance-based assessment (FDA-NIH Biomarker Working Group, 2016). A clinical outcome assessment may be used in clinical studies to provide either direct or indirect evidence of treatment benefit. PRO is a type of clinical outcome assessment. PRO is an umbrella term referring to all outcomes and symptoms that are directly reported by the patient. PROs have become important in evaluating effectiveness of study treatments in clinical studies and will aid in understanding of the benefit/risk evaluation (Kluetz et al., 2018). The following PROs will be administered in this study: EORTC-QLQ-C30, EORTC-QLQ-BIL21, PRO-CTCAE, PGIS, and EQ-5D-5L. Each is described below. PROs will be translated into the language of the country in which they are being administered, with the exception of PRO-CTCAE that will only be administered in languages where a linguistically validated version is available.

### EORTC QLQ-C30 and EORTC QLQ-BIL21

The EORTC-QLQ-C30 and the EORTC QLQ-BIL21 are selected to assess the impact of treatment and disease state on symptoms, impacts, and HRQOL. The EORTC scales include many of the key BTC symptoms and impacts, such as abdominal pain, fatigue, pruritus, jaundice, lack of appetite, physical functioning, and insomnia.

The EORTC QLQ-C30 includes 30 items and assesses HRQOL/health status through the following 9 multi-item scales: 5 functional scales (i.e., physical, role, cognitive, emotional, and social), 3 symptom scales (i.e., fatigue, pain, and nausea and vomiting), and 1 global health status/QoL scale. It also includes 6 single-item symptom/impact measures: dyspnea, insomnia, appetite loss, constipation, diarrhea, and financial difficulties (Aaronson et al., 1993).

The EORTC QLQ-BIL21 was developed to measure QOL in patients with BTC and is used as an accompaniment to the EORTC QLQ-C30. The EORTC QLQ-BIL21 consists of 21 questions: 3 single-item assessments relating to treatment side effects, difficulties with drainage bags/tubes, and concerns regarding weight loss, in addition to 18 items grouped into 5 scales, which are pain symptoms (4 items), tiredness symptoms (3 items), jaundice symptoms (3 items), anxiety symptoms (4 items), and eating symptoms (4 items).

The EORTC QLQ-BIL21 has evidence of content validity in BTC patients. It was developed based on a literature review, in-depth patient interviews, and healthcare provider interviews specific to BTC populations (Friend et al., 2011). Its measurement properties have also been tested in BTC populations and include good evidence of internal consistency reliability, test-retest reliability (reproducibility), and construct validity (Kaup-Robberts et al., 2016).

### PRO-CTCAE

The PRO-CTCAE is included to address tolerability from the patients' perspective. It was developed by the National Cancer Institute (NCI). It was developed in recognition that collecting treatment-related symptom data directly from patients can improve accuracy and efficiency. This was based on findings from multiple studies demonstrating that physicians and nurses underestimate treatment-related symptom onset, frequency, and severity in comparison with patient ratings (Basch et al., 2009; Litwin et al., 1998; Sprangers and Aaronson, 1992). The PRO-CTCAE is an item-bank of symptoms, and the items have undergone extensive qualitative review among experts and patients. To date, 78 symptoms of the CTCAE have been identified to be amenable to patient reporting, but not all items are administered in any one clinical study. Six different symptomatic AEs were selected for the PRO-CTCAE based on available safety reports of durvalumab and gemcitabine/cisplatin. Generally, symptomatic AEs that were not included in the EORTC were prioritized for the PRO-CTCAE to reduce patient burden per published recommendations (Trask et al., 2018). The PRO-CTCAE will only be administered in the languages where a linguistically validated version exists.

### PGIS

The PGIS item is included to assess how a patient perceives his/her overall severity of cancer symptoms over the past 7 days. This is a single-item questionnaire, and patients will choose from response options ranging from “no symptoms” to “very severe.”

### EQ-5D-5L

The EQ-5D-5L, developed by the EuroQOL Group, is a generic questionnaire that provides a simple descriptive profile of health and a single index value for health status for economic appraisal (EuroQol Group, 2015). The EQ-5D-5L questionnaire comprises 5 questions that cover 5 dimensions of health (i.e., mobility, self-care, usual activities, pain/discomfort, and anxiety/depression). Respondents also assess their health today using the EQ-visual analog scale (VAS), which ranges from 0 (worst imaginable health) to 100 (best imaginable health).

### Disease-Specific Tumor Marker Samples

As part of the routine safety blood samples, carcinoembryonic antigen (CEA) and carbohydrate antigen 19-9 (CA-19-9) assessment will take place. It is important to follow the assessment schedule as closely as possible. A rise in CEA or CA-19-9 alone is not sufficient to declare progression and discontinue treatment.

Progression events should be determined by radiographic evidence of progression based on RECIST 1.1.

Further assessment of CEA and CA-19-9 after radiological progression will be at the discretion of the Investigator according to local clinical practice.

### Exploratory Genetic Testing

Buffy coat layers obtained during plasma isolation, may be retained and analysed for germline mutations and/or in identification of tumor-specific mutations for Microsatellite instability (MSI)/tumor mutational burden (TMB) assessment to distinguish more accurately tumor-specific mutations, according to local regulations.

### Biomarkers

Tissue samples will be obtained from all screened patients.

Pretreatment tumor PD-L1 expression will be evaluated (retrospectively) in all randomized patients. Data will be compared between arms to determine if baseline PD-L1 status is prognostic and/or predictive of outcomes associated with Arm A versus Arm B. Baseline tumor requirements are briefly described herein.

Based on availability of tissue, additional exploratory biomarkers may be evaluated as described herein.

The results may be pooled with biomarker data from other durvalumab studies to evaluate biological responses across indications and to compare results in monotherapy versus combination settings.

### PD-L1 Expression Assessment

The Ventana PD-L1 IHC assay will be used to determine PD-L1 status in the tumor samples.

### Exploratory Biomarkers

Blood and tumor samples for exploratory biomarker analyses will be obtained.

Baseline measurements and/or changes in measurements in on-treatment samples from baseline will be correlated with outcomes and/or drug treatment. Note that samples will be obtained from patients randomized to each treatment arm. Comparisons will be made between baseline measures to determine if biomarkers (or combination of markers) are prognostic or predictive of outcomes.

### Microsatellite Instability (MSI) and Tumor Mutation Burden (TMB)

Tumor tissue remaining after PD-L1 testing may be analyzed retrospectively for microsatellite instability (MSI) using a Next Generation Sequencing (NGS)-based method such as whole exome sequencing (WES) or a targeted NGS assay. The MSI-H/MSS status and TMB will be determined based on the MSI events or somatic variants detected in the tumor sequence data and correlated with efficacy as an exploratory objective. Blood-based MSI or TMB testing may also be performed in cases of insufficient tissue material, unsuccessful tissue-based MSI testing or for evaluation of blood-based MSI/TMB with clinical outcomes and other biomarkers. The matched normal DNA obtained from buffy coat layer collected during plasma isolation may be used in identification of tumor-specific mutations for MSI/TMB assessment. The incorporation of a matched normal DNA in the mutation analysis helps distinguish more accurately tumor-specific mutations in heterogeneous tumor cell populations and thus may also reduce or prevent unintentional inclusion of normal inherited germline mutations in the final analysis or study reports.

In the event NGS-based MSI testing is not feasible or fails, MMR testing by IHC or similar methods will be performed.

### Tumor Microenvironment Biomarkers

Based on the availability of tissue, a panel of additional immune-relevant markers expressed in TILs or tumor cells may be assessed using immunohistochemistry (IHC), gene expression profiling or other technically-feasible methods. Markers of special interest include, but are not limited to, CD8, FOXP3, CD68, CD3, CD73, NKp46, CXCL9 and LAG3.

Other tissue-based approaches may be pursued including but not limited to neo-antigen prediction, T cell receptor clonality assessment, molecular profiling and/or somatic variant detection methodologies. Note: This analysis will not be conducted in China.

### Whole Blood Gene Expression (PaxGene RNA)

Whole blood samples will be obtained for RNA analysis. Total RNA will be prepared for quantification of RNA, micro-RNA, and/or other non-coding RNA using reverse transcription quantitative polymerase chain reaction, microarray, sequencing, or other technology. Focus is likely to be given to the expression of immunomodulatory genes. Baseline and/or on-treatment correlations with outcome data will be completed on select candidate predictive markers, with the aim of identifying useful expression thresholds for identifying patients likely to receive benefit. These samples may also be used for other molecular analyses of features associated with immune responses such as, but not limited to, T cell receptor clonality and HLA type. Note: This sample will not be collected in China.

### Soluble Factors-Plasma

Blood samples will be collected at multiple timepoints for analysis of circulating factors such as circulating tumor deoxyribonucleic acid (ctDNA) in plasma. Analyses may include, but not be limited to evaluating baseline sensitizing mutations to treatment and correlations with clinical outcomes, changes in levels and variant frequencies of ctDNA and potentially minimal residual disease. Plasma may also be evaluated for relevant cytokines, chemokines and other soluble biomarkers.

The buffy coat layer obtained during the plasma isolation process of the baseline sample may be retained and analyzed per local regulations. DNA isolated from the buffy coat will be used as a patient-specific normal DNA reference to determine whether mutations identified in ctDNA or tumor mutational analysis are tumor-specific (somatic). Note: This analysis will not be conducted in China.

### Serum Sample for Biomarkers

Serum will be obtained to explore the expression of cytokines and chemokines, including but not limited to IFN-γ, interleukin 18, CXCL9, and CXCL10. Similarly, the concentrations of a battery of IC ligands, receptors, or other soluble factors may be assessed. Proteins of special interest include CTLA-4, PD-1, PD-L1, B7-1, B7-2, and IL6R. In addition, serum proteome may be explored using mass-spectrometry based or similar technologies. Circulating exosomes may also be isolated from serum to evaluate exosome RNA, DNA, and/or protein contents. Note: This sample will not be collected in China.

### Outcome Measures for Analyses

### Statistical Methods

The primary objective of the study is to confirm the superiority of Arm A compared to Arm B in terms of OS in patients with advanced BTC.

The primary endpoint OS is defined as the time from the date of randomization until death due to any cause. Secondary efficacy endpoints include progression-free survival (PFS), ORR, duration of response (DoR), disease control rate (DCR), and PRO measures.

The Full Analysis Set (FAS) will include all randomized patients. The FAS will be used for all efficacy analyses, including PROs. Treatment groups will be compared on the basis of randomized study treatment, regardless of the treatment actually received. Patients who were randomized but did not subsequently go on to receive study treatment are included in the analysis in the treatment group to which they were randomized.

A hypothesis of improved OS will be tested when:

Approximately 397 OS events have occurred across Arm A and Arm B (59% maturity) Interim Analysis 2 (IA-2) AND

Approximately 496 OS events have occurred across Arm A and Arm B (74% maturity) final analysis (FA).

The primary analysis of OS is based on log-rank test for interim analysis and Fleming-Harrington (FH) (0, 1) test for final analysis. A sensitivity analysis using the log-rank test will also be performed at the final analysis. If the true average OS hazard ratio (HR) is 0.745, approximately 496 OS events will provide 90% power to demonstrate statistical significance at the 4.20% level (using a 2-sided log-rank test), allowing for 1 interim analysis for clinical activity assessed by ORR and DoR and 1 interim analysis for early testing for superiority in OS improvement at approximately 80% of target events. The smallest treatment difference that could be statistically significant at the FA is an average HR of 0.833 using log-rank test. With a 21-month recruitment period and a minimum follow-up period of 19 months assumed, it is anticipated that this analysis will be performed approximately 40 months after the first patient is randomized. With log-rank test for the second interim analysis and FH (0, 1) as the final analysis, the overall power is at least 86% under various scenarios considered.

Two interim analyses and 1 FA are planned for the evaluation of efficacy:

IA-1: The objective of IA-1 is to assess clinical activity. ORR and DoR will be summarized to support early registration of durvalumab when administered in combination with gemcitabine/cisplatin. The summary will be done both for Investigator assessments and for blinded independent central review (BICR) assessments according to RECIST 1.1. The planned data cutoff (DCO) for IA-1 will occur when at least 200 patients have had the opportunity to be followed for at least 32 weeks or the last patient has been randomized to the global cohort, whichever comes later. The analysis set will include all randomized patients who have had the opportunity for at least 32 weeks of follow-up at the time of the IA-1 DCO (i.e., randomized ≥ 32 weeks prior to IA-1 DCO).

Based on enrollment assumptions, it is expected that this will occur approximately 21 months after randomization of the first patient.

IA-2: The second interim analysis will test for early superiority of the durvalumab regimen relative to control. This analysis will be performed when approximately 397 OS events have been observed in the study (59% maturity or 80% information fraction with respect to log-rank test) with 75% power. Based on enrollment assumptions, it is expected that this will occur approximately 31 months after randomization of the first patient.

IA-2 will evaluate the efficacy of Arm A compared to Arm B in terms of OS (primary objective). OS will be analyzed using a stratified log-rank test (stratified by disease status and primary tumor location). The treatment effect will be estimated by the HR, 95% confidence interval (CI), and p-value.

A small alpha expenditure of 0.001 (0.1%) will be allocated to IA-1. Strong control of the familywise error rate (FWER) at the remaining 4.9% level (2 sided) across the testing of OS and PFS endpoints will be achieved through a combined approach of alpha allocation to the OS analyses (IA-2 and the FA) via alpha spending function and a hierarchical testing procedure; that is, PFS will be tested only if OS met statistical significance at IA-2 or FA (Glimm et al., 2010). The IA-2 for OS will be conducted when approximately 397 of the 496 expected OS events (i.e., 80% information fraction with respect to log-rank test) have occurred. Using the Lan-DeMets spending function approximating O'Brien-Fleming boundaries, 2-sided significance levels of 0.0238 and 0.0420 will be applied to OS IA-2 and FA, respectively using log-rank test (Lan and DeMets, 1983). The significance level of FH (0, 1) test at the final analysis will be determined based on the correlation between interim data and final data (Tsiatis, 1982).

PFS will be formally tested using PFS information collected up to each DCO if OS meets statistical significance at that DCO (IA-2 or FA). Significance levels for PFS at IA-2 and FA will be derived based on the alpha spending function approximating Pocock boundaries, which strongly controls the Type I error at the 0.049 level (2-sided). Assuming approximately 506 PFS events and 590 PFS events are available at the time of each PFS analysis, PFS testing will be carried out with 2-sided significance levels of 0.0444 and 0.0236 at IA-2 and FA based on log-rank test, respectively. Since DCO timing will be determined based on the number of OS events, the nominal significance level for PFS analysis might be adjusted for the actual information fraction for PFS at IA-2 relative to FA.

Safety data will be summarized descriptively and will not be formally analyzed.

Additional exploratory overall survival analyses will be performed using long-term follow-up survival data. Long-term survival data may be collected for up to approximately 4 years after the last patient randomization in Global cohort.

### Populations for Analyses

Definitions of the analysis sets for each outcome variable are provided in Table 4.

TABLE 4
Summary of outcome variables and analysis populations

| | |
|---|---|
| Outcome variable | Populations |

Efficacy Data

| | |
|---|---|
| OS | Full Analysis Set |
| PFS | Full Analysis Set |
| ORR, DoR, and PRO endpoints | Full Analysis Set |
| Demography and other baseline characteristics | Full Analysis Set |
| PK data | PK Analysis Set |

Biomarker data

| | |
|---|---|
| Biomarker data | Full Analysis Set |

Safety Data

| | |
|---|---|
| Exposure | Safety Analysis Set |
| AEs | Safety Analysis Set |
| Laboratory measurements | Safety Analysis Set |
| Vital Signs and other safety | Safety Analysis Set |
| ADA data | ADA Analysis Set |

AE Adverse event; DoR Duration of response; ORR Objective response rate; OS Overall survival; PFS Progression-free survival; PK Pharmacokinetics; PRO Patient-reported outcome.

### Full Analysis Set

The FAS will include all randomized patients. The FAS will be used for all efficacy analyses, including PROs. Treatment groups will be compared on the basis of randomized study treatment, regardless of the treatment actually received. Patients who were randomized but did not subsequently go on to receive study treatment are included in the analysis in the treatment group to which they were randomized.

Analysis for ORR will be based on patients in the FAS, who had a measurable disease at baseline. Analysis of DoR will be based on patients in the FAS who achieved objective response.

### Safety Analysis Set

The SAS will consist of all patients who received at least 1 dose of study treatment. Safety data will not be formally analyzed but summarized descriptively using the SAS according to the treatment received; that is, erroneously treated patients (e.g., those randomized to treatment A but actually given treatment B) will be summarized according to the treatment they actually received.

### Pharmacokinetic Analysis Set

All patients who receive at least 1 dose of durvalumab per the protocol for whom any post-dose data are available and who do not violate or deviate from the protocol in ways that would significantly affect the PK analyses will be included in the PK Analysis Set. The population will be defined by the Study Physician, Pharmacokineticist, and Statistician prior to any analyses being performed.

### ADA Analysis Set

The ADA analysis set will include all subjects who have non-missing baseline ADA and at least 1 non-missing post-baseline ADA results. All major ADA analyses will be based on the ADA analysis set.

### Safety Assessments

For the change from baseline summaries for vital signs, laboratory data, ECGs, and physical examinations, the baseline value will be the latest result obtained prior to the start of study treatment.

QTcF will be derived during creation of the reporting database using the reported ECG values (RR and QT) using the following formula:

QTcF=QT/RR^{1/3}, where RR is in seconds

Corrected calcium product will be derived during creation of the reporting database using the following formula:

Corrected calcium(mmol/L)=Total calcium(mmol/L)+([40−albumin(G/L)]×0.02)

The denominator used in laboratory summaries will only include evaluable patients, i.e., those who had sufficient data to have the possibility of an abnormality.

For example:

If a CTCAE criterion involves a change from baseline, evaluable patients would have both a pre-dose and at least 1 post-dose value recorded.

If a CTCAE criterion does not consider changes from baseline to be evaluable, the patient need only have 1 post-dose value recorded.

The denominator in vital signs data should include only those patients with recorded data.

### Calculation or Derivation of Patient-Reported Outcome Variables

### EORTC QLQ-C30

The EORTC QLQ-C30 consists of 30 questions that can be combined to produce 5 functional scales (physical, role, cognitive, emotional, and social), 3 symptom scales (fatigue, pain, and nausea/vomiting), and global health status/QOL scale. The EORTC QLQ-C30 will be scored according to the EORTC QLQ-C30 Scoring Manual (Fayers et al., 2001). An outcome variable consisting of a score from 0 to 100 will be derived for each of the symptom scales, each of the functional scales, and the global measure of health status scale in the EORTC QLQ-C30 according to the EORTC QLQ-C30 Scoring Manual. Higher scores on the global measure of health status and functional scales indicate better health status/function, but higher scores on symptom scales represent greater symptom severity. For each subscale, if <50% of the subscale items are missing, then the subscale score will be divided by the number of non-missing items and multiplied by the total number of items on the subscales (Fayers et al., 2001). If at least 50% of the items are missing, then that subscale will be treated as missing. Missing single items are treated as missing. The reason for any missing questionnaire will be identified and recorded.

### Definition of Clinically Meaningful Changes-Visit Response and Best Overall Response

The global health status/QOL scale includes 2 items from the EORTC QLQ-C30: “How would you rate your overall health during the past week?” (item 29) and “How would you rate your overall QOL during the past week?” (item 30). Definition of clinically meaningful changes in score compared with baseline will be evaluated. A clinically meaningful change is defined as an absolute change in the score from baseline of ≥10 for scales from the EORTC QLQ-C30 (Osoba et al., 1998). For example, a clinically meaningful improvement in physical function (as assessed by EORTC QLQ-C30) is defined as an increase in the score from baseline of ≥10, whereas a clinically meaningful deterioration is defined as a decrease in the score from baseline of ≥10. At each post-baseline assessment, the change in global health status/QOL, symptoms, and functioning score from baseline will be categorized as improvement, no change, or deterioration as shown in Table 5.

TABLE 5
Mean change and clinically meaningful
change - EORTC QLQ-C30

| Score | Change from baseline | Visit response |
|---|---|---|
| EORTC QLQ-C30 | ≥10-point increase | Improvement |
| global quality-of- | ≥10-point decrease | Deterioration |
| life score | or “Patient too sick to complete | |
| | the questionnaires (disease | |
| | under investigation)” | |
| | Otherwise | No change |
| EORTC QLQ-C30 | ≥10-point increase | Deterioration |
| symptom score | or “Patient too sick to complete | |
| | the questionnaires (disease | |
| | under investigation)” | |
| | ≥10-point decrease | Improvement |
| | Otherwise | No change |
| EORTC QLQ-C30 | ≥10-point increase | Improvement |
| functional score | ≥10-point decreaseor | Deterioration |
| | “Patient too sick to complete | |
| | the questionnaires (disease | |
| | under investigation)” | |
| | Otherwise | No change |

A patient's best overall response in symptoms, function, or global health status/QOL will be derived as the best response the patient achieved based on evaluable PRO data collected during the study period. The criteria in Table 6 will be used to assign a best response in symptoms, function, or global health status/QoL.

TABLE 6
Best response in EORTC QLQ-C30 and EORTC QLQ-BIL21 scores: FAS

| Overall score | |
|---|---|
| response | Criteria |
| Missing | Patient has no evaluable baseline or post-baseline PRO assessment. |
| Improved | Patient meets one of the following criteria: |
| | Has 2 consecutive visit responses of “improvement” at least 14 days apart |
| | Has 1 visit response of “improvement” with no further assessments and |
| | did not die within 2 PRO assessment visits |
| No Change | Patient does not qualify for an overall score response of “improved” and meets |
| | one of the following criteria: |
| | 1 Has 2 consecutive visit responses of “no change” at least 14 days apart |
| | Has 1 visit response of “no change” with no further assessments and did not |
| | die within 2 PRO assessment visits |
| Deterioration | Patient does not qualify for an overall score response of “improved” or “no |
| | change” and meets one of the following criteria: |
| | 1 Has 2 consecutive visit responses of “deterioration” at least 14 days apart |
| | Has 1 visit response of “deterioration” and no further assessments |
| | Has 1 visit response of “improvement” or “no change” followed by death |
| | within 2 PRO assessment visits |
| Other | Does not qualify for one of the above (improved, no change, or deterioration). |

EORTC European Organisation for Research and Treatment of Cancer; FAS Full analysis set; PRO Patient-reported outcome; QLQ-C30 30-item core quality-of-life questionnaire; QLQ-BIL21 21-item Cholangiocarcinoma and Gallbladder Cancer Quality of Life Questionnaire.

### Time to HRQOL or Function Deterioration

Time to QoL or function deterioration will be defined as the time from the date of randomization until the date of the first clinically meaningful deterioration that is confirmed at a subsequent visit (except if it was the patient's last available assessment) or death (by any cause) in the absence of a clinically meaningful deterioration, regardless of whether the patient discontinues the study treatment(s) or receives another anticancer therapy prior to global health status/QOL or function deterioration. Death will be included as an event only if it occurs within 2 PRO assessment visits from the last available PRO assessment. A sensitivity analysis will be conducted, in which deaths will be censored at the date of death.

Patients whose global health status/QOL or function (as measured by EORTC QLQ-C30) have not shown a clinically meaningful deterioration and who are alive at the time of the analysis will be censored at the time of their last PRO assessment, where the global health status/QOL or function could be evaluated. Also, if global health status/QOL or function deteriorates or the patient dies after 2 or more missed PRO assessment visits, the patient will be censored at the time of the last PRO assessment, where global health status/QOL or function could be evaluated prior to the 2 missed visits.

The population for the analysis of time to global health status/QOL or function deterioration will include a subset of the FAS who have baseline scores of ≥10.

### Symptom Improvement Rate

Responses in symptoms for each visit (improvement, deterioration, and no change) based on Table 6 as well as the best overall response will be presented by treatment group. The symptom improvement rate will be defined as the number (%) of patients with a best overall score response of “improved” in symptoms.

The denominator will consist of a subset of the FAS who have a baseline symptom score ≥10.

### Global Health Status/QoL or Function Improvement Rate

The global health status/QOL or function improvement rate will be defined as the number (%) of patients with a best overall response of “improved” in QoL or function.

The denominator will consist of a subset of the FAS who have a baseline global health status/QOL or function score≤90.

### EORTC QLQ-BIL21

The QLQ-BIL21 is a BTC-specific module from the EORTC comprising 21 questions to assess BTC symptoms. The module includes 5 multi-item domain scales and 3 single-item scales. For all items and scales, high scores indicate increased symptomatology/more problems.

The scoring approach for the QLQ-BIL21 is identical in principle to that for the symptom scales/single items of the EORTC QLQ-C30. Similar to the symptom scales of the EORTC QLQ-C30, higher scores represent greater symptom severity.

### Definition of Clinically Meaningful Change-Visit Response and Best Overall Response

Changes in score compared with baseline will be evaluated. A clinically meaningful change is defined as an absolute change in the score from baseline of >10 for scales/items from QLQ-BIL21. For example, a clinically meaningful deterioration or worsening in pain (as assessed by QLQ-BIL21) is defined as an increase in the score from baseline of ≥10. At each post-baseline assessment, the change in symptom score from baseline will be categorized as improved, no change, or deterioration, as shown in Table 7. A patient's best overall response in symptoms will be derived as the best response the patient achieved based on evaluable PRO data collected during the study period. The criteria in Table 6 will be used to assign a best response in symptom score.

TABLE 7
Mean change and clinically meaningful
change - EORTC QLQ-BIL21

| Score | Change from baseline | Visit response |
|---|---|---|
| QLQ-BIL21 symptom | ≥+10 (increase of at least 10) | Deterioration |
| scales and items | or “Patient too sick to complete | |
| | the questionnaires (disease under | |
| | investigation)” | |
| | ≥−10 (decrease of at least 10) | Improved |
| | Otherwise | No change |

EORTC European Organisation for Research and Treatment of Cancer; QLQ-BIL21 21-item Cholangiocarcinoma and Gallbladder Cancer Quality of Life Questionnaire.

### Time to Symptom Deterioration

For each of the symptom scales/items in the QLQ-BIL21, time to symptom deterioration will be defined as the time from randomization until the date of the first clinically meaningful symptom deterioration that is confirmed at a subsequent visit (except if it was the patient's last available assessment) or death (by any cause) in the absence of a clinically meaningful symptom deterioration, regardless of whether the patient discontinues the study treatment(s) or receives another anticancer therapy prior to symptom deterioration. Only deaths occurring within 2 PRO assessment visits from the last available PRO assessment will be included as events. A sensitivity analysis will be conducted in which deaths will be censored at the date of death.

Patients whose symptoms (as measured by the QLQ-BIL21) have not shown a clinically meaningful deterioration and who are alive at the time of the analysis will be censored at the time of their last PRO assessment, where the symptom could be evaluated. Also, if symptoms progress or the patient dies after 2 or more missed PRO assessment visits, the patient will be censored at the time of the last PRO assessment, where the symptom could be evaluated prior to the 2 missed visits.

The population for the analysis of time to symptom deterioration will include a subset of the FAS who have baseline scores≤90.

### EQ-5D-5L

The EQ-5D-5L questionnaire comprises 6 questions that cover 5 dimensions of health (e.g., mobility, self-care, usual activities, pain/discomfort, and anxiety/depression) and a VAS. For each dimension, respondents select which statement best describes their health on that day from a possible 5 options of increasing levels of severity (e.g., no problems, slight problems, moderate problems, severe problems, and unable to/extreme problems).

A unique EuroQOL 5-dimension (EQ-5D) health state, termed the EQ-5D-5L profile, is reported as a 5-digit code with a possible 3125 health states based on these responses. For example, state 11111 indicates no problems on any of the 5 dimensions. This EQ-5D-5L profile will be converted into a weighted health state utility value, termed the EQ-5D index, by applying a country-specific equation to the profile that represents the comparative value of health states. This equation is based on national valuation sets elicited from the general population, and the base case will be the UK perspective. Where a valuation set has not been published, the EQ-5D-5L profile will be converted to the EQ-5D index using a crosswalk algorithm (van Hout et al., 2012).

The evaluable population will comprise a subset of the FAS who have a baseline EQ-5D-5L assessment.

### PGIS

PGIS data will be presented using summaries and descriptive statistics. Additionally, PGIS data will be further explored to support anchor-based analyses of clinically meaningful change supplemented with empirical cumulative distribution function and probability density function curves.

### PRO-CTCAE

The PRO-CTCAE consists of nominal categories (e.g., “none” to “very severe” for some items in the questionnaire).

### Calculation or Derivation of Pharmacokinetic Variables

### Population Pharmacokinetics and Exposure-Response/Safety Analysis

A population PK model will be developed using a non-linear mixed-effects modeling approach. The impact of physiologically relevant patient characteristics (covariates) and disease on PK will be evaluated. The relationship between the PK exposure and the effect on safety and efficacy endpoints will be evaluated. The results of such an analysis will be reported in a separate report. The PK, demographic, safety, and efficacy data collected in this study may also be combined with similar data from other studies and explored using population PK and/or exposure-response/safety analysis.

### Pharmacokinetic Analysis

Non-compartmental analysis will not be performed to calculate PK parameters due to sparse sampling. Durvalumab serum concentration data and summary statistics will be tabulated. Individual and mean serum concentration-time profiles will be generated. Samples below the lower limit of quantification will be treated as missing in summary statistics.

### Immunogenicity Analysis

Immunogenicity results will be analyzed descriptively by summarizing the number and percentage of patients who develop detectable ADAs against durvalumab. The immunogenicity titer and presence of neutralizing ADAs will be reported for samples confirmed positive for the presence of ADAs. The effect of immunogenicity on PK, efficacy, and safety will be evaluated, if the data allow.

### Calculation or Derivation of Biomarker Variables

PD-L1 status, as defined in the secondary objectives, will be assessed for evaluable patients in each cohort.

### Calculation or Derivation of Pharmacogenetic Variables

In the case of genetic data, only the date that the patient gave consent to participation in the genetic research and the date the blood sample was taken from the patient will be recorded in the eCRF and database.

### Primary Endpoint: Overall Survival

OS in the FAS will be analyzed using a stratified log-rank test, adjusting for disease status (initially unresectable or recurrent) and primary tumor location (intrahepatic cholangiocarcinoma, extrahepatic cholangiocarcinoma, or gallbladder cancer) at IA-2 and using stratified FH (0, 1) test at FA. The effect in Arm A versus Arm B will be estimated by the HR together with its corresponding CI and p-value. Kaplan-Meier plots will be presented by treatment arm. Summaries of the numbers and percentages of patients who have died, those still in survival follow-up, those lost to follow-up, and those who have withdrawn consent will be provided along with the mOS for each treatment.

Cox proportional hazards model will be used to estimate HR and corresponding CI based on Long-term follow up OS data. KM plots of Overall Survival will also be presented by treatment arm. Additional analysis may be performed if appropriate.

### Sensitivity Analysis

The assumption of proportionality will be assessed first by examining the plots of complementary log-log (event times) versus log (time) and, if these raise concerns, by fitting a time-dependent covariate to assess the extent to which this represents random variation. If a lack of proportionality is evident, the variation in treatment effect will be described by presenting piecewise HR calculated over distinct time-periods. In such circumstances, the HR can still be meaningfully interpreted as an average HR over time unless there is extensive crossing of the survival curves. If lack of proportionality is found, this may be a result of treatment-by-covariate interactions, which will be investigated. In addition, the Kaplan Meier (KM) curve along with landmark analyses (e.g., 1-year OS rate) will also help in understanding the treatment benefit.

Attrition bias will be investigated by a KM plot of the time to censoring using the OS data from the primary analysis and where the censoring indicator of the OS analysis is reversed.

Cox proportional hazards modeling will be used to assess the effect of covariates on the HR estimate. A model will be constructed, containing treatment and the stratification factors, to ensure that any output from the Cox modeling is likely to be consistent with the results of the stratified log-rank test.

Interactions between treatment and stratification factors will also be tested to rule out any qualitative interaction using the approach of Gail and Simon, 1985.

At FA OS will be analyzed using a stratified log-rank test, adjusting for disease status (initially unresectable or recurrent) and primary tumor location (intrahepatic cholangiocarcinoma, extrahepatic cholangiocarcinoma, or gallbladder cancer) to ensure that any output from the stratified log-rank test is likely to be consistent with the methods of the FH (0, 1) test.

Delayed effect, i.e., delayed separation of survival curves is expected in immunotherapy trials, and has been noted in the majority of IO+chemo vs chemo Phase III trials reported to date. The strategy of using log-rank test at the interim analysis and FH (0, 1) test at final analysis provides robust statistical power and inference under various scenarios including proportional hazards and delayed effects. Log-rank test is the most powerful approach in the scenario of proportional hazards and FH (0, 1) is more powerful in the scenario of delayed effects. In addition, at the final analysis, the data maturity is high and consequently the weight function of FH (0, 1) test is stable.

### Subgroup Analysis

Subgroup analyses will be conducted comparing OS between Arm A versus Arm B in the following subgroups of the FAS (but not limited to):
Sex (male versus female)
Age at randomization (<65 versus ≥65 years of age).
PD-L1 status
Disease status (initially unresectable versus recurrent).
Primary tumor location (intrahepatic cholangiocarcinoma versus extrahepatic cholangiocarcinoma versus gallbladder cancer).
Race (Asian versus non-Asian)

Other baseline variables may also be assessed if there is clinical justification or an imbalance is observed between the treatment arms. The purpose of the subgroup analyses is to assess the consistency of treatment effect across expected prognostic and/or predictive factors. Forest plots will be presented.

Additionally, for each subgroup, the HR (Arm A:Arm B) and 95% CI will be calculated from a Cox proportional hazards model with treatment as the only covariate. These will be presented on a forest plot including the HR and 95% CI from the overall population.

No adjustment to the significance level for testing of the subgroup and sensitivity analyses will be made, since all these analyses will be considered supportive of the analysis of OS and PFS.

### Progression-Free Survival

The secondary PFS analysis will also be based on the programmatically derived RECIST 1.1 using the Investigator tumor assessments. The analysis will be performed in the FAS using a stratified log-rank test, adjusting for disease status and primary tumor location. The effect of Arm A versus Arm B will be estimated by the HR together with its corresponding 95% CI and p-value.

Kaplan-Meier plots of PFS will be presented by treatment arm. Summaries of the number and percentage of patients experiencing a PFS event and the type of event (RECIST 1.1 or death) will be provided along with median PFS for each treatment.

### Sensitivity Analysis

Sensitivity analyses will be performed to assess possible evaluation-time bias that may be introduced if scans are not performed at the protocol-scheduled timepoints. The midpoint between the time of progression and the previous evaluable RECIST assessment will be analyzed using a log-rank test. For patients whose death was treated as PFS event, the date of death will be used to derive the PFS time used in the analysis. This approach has been shown to be robust even in highly asymmetric assessment schedules (Sun and Chen, 2010).

Attrition bias will be assessed by repeating the PFS analysis except that the actual PFS event times, rather than the censored times, of patients who progressed or died in the absence of progression immediately following 2 or more non-evaluable tumor assessments will be included. In addition, patients who take subsequent therapy prior to progression or death will be censored at their last evaluable assessment prior to taking the subsequent therapy. This analysis will be supported by a Kaplan-Meier plot of the time to censoring where the censoring indicator of the PFS analysis is reversed.

The assumption of proportionality will be assessed first by examining the plots of complementary log-log (event times) versus log (time) and, if these raise concerns, by fitting a time-dependent covariate to assess the extent to which this represents random variation. If a lack of proportionality is evident, the variation in treatment effect will be described by presenting piecewise HR calculated over distinct time-periods. In such circumstances, the HR can still be meaningfully interpreted as an average HR over time unless there is extensive crossing of the survival curves. If lack of proportionality is found, this may be a result of treatment-by-covariate interactions, which will be investigated. In addition, the Kaplan-Meier curve along with landmark analyses (e.g., 1-year PFS rate) will also help in understanding the treatment benefit.

Cox-proportional hazards modeling will be used to assess the effect of covariates on the HR estimate. A model will be constructed, containing treatment and the stratification factors, to ensure that any output from the Cox modeling is likely to be consistent with the results of the stratified log-rank test.

Interactions between treatment and stratification factors will also be tested to rule out any qualitative interaction using the approach of (Gail and Simon, 1985).

Further sensitivity analysis may be documented in the SAP.

### Subgroup Analysis

Subgroup analyses will be conducted comparing PFS (per RECIST 1.1 using Investigator assessments) between Arm A and Arm B in the subgroups of the FAS.

### Objective Response Rate

ORR will be based on the programmatically derived RECIST using the Investigator tumor data. ORR will be compared between Arm A and Arm B using a stratified CMH test adjusting for the same factors as the primary endpoint (disease status and primary tumor location). The results of the analysis will be presented in terms of a p-value. This analysis will be performed in the subset of patients in the FAS who had a measurable disease at baseline.

Summaries will be produced that present the number and percentage of patients with a tumor response (CR/PR). Overall visit response data will be listed for all patients (i.e., the FAS). For each treatment arm, best overall response (BoR) will be summarized by n (%) for each category (CR, PR, SD, PD, and NE). No formal statistical analyses are planned for BoR.

At IA-1, the ORR evaluation will be repeated with tumor data as assessed by BICR according to RECIST 1.1 for the randomized patients who have had the opportunity to be followed for at least 32 weeks.

### Duration of Response

KM plots of DoR based on the Investigator assessment of RECIST 1.1 will be presented. Median DoR will also be summarized and calculated from the KM curve. Only patients who have a confirmed response will be included in this summary table. Swimmer plots that clearly show the profile of each patient who responds will also be produced.

At IA-1, the DoR evaluation will be repeated with tumor data as assessed by BICR according to RECIST 1.1 for the randomized patients who have had the opportunity to be followed for at least 32 weeks.

### Disease Control Rate

The DCR, DCR-24w, and DCR-32w based on Investigator assessments per RECIST 1.1 will be summarized (i.e., number of patients [%]) per treatment arm.

### Patient-Reported Outcomes

### EORTC QLQ-C30

The primary assessment of symptoms, impacts, and HRQOL will focus on time to deterioration (TTD), which will be analyzed using a stratified log-rank test as described for the PFS endpoint. Separate analyses will be conducted for global health status/QOL, function (physical, role, cognitive, social, and emotional), multi-term symptoms (fatigue and pain), and single items (appetite loss and insomnia). The effect of durvalumab therapy versus placebo will be estimated by the HR together with its corresponding CI and p-value. Kaplan-Meier plots will be presented by treatment group. Summaries of the number and percentage of patients who have an event as well as who were censored will be provided along with the medians for each treatment.

Additional analyses of HRQOL, impacts, and symptoms will focus on comparing mean change from baseline in the global health status/QOL, functions (physical, role, cognitive, social, and emotional), multi-term symptoms (fatigue and pain), and single items (appetite loss and insomnia) score between treatment groups. The analysis population for mean change in HRQOL, impacts, or symptoms data will be the FAS and will include all randomized patients with an evaluable baseline assessment and at least 1 evaluable post-baseline assessment. Change from baseline will be derived using a mixed model repeat measures (MMRM) analysis of all the post-baseline scores for each visit. The model will include treatment, visit, and treatment-by-visit interaction as explanatory variables and the baseline score as a covariate. Adjusted mean change from baseline estimates per treatment group and corresponding 95% CIs will be presented along with an overall estimate of the treatment difference, 95% CI, and p-value.

Summary tables of visit responses for each EORTC QLQ-C30 scale/item score (global health status/QoL, 5 functions, and all symptoms) and for each visit (improvement, deterioration, and no change) will be presented by treatment group. In addition, summary tables of the best overall response will be provided for the following domains by treatment group: global health status/QOL, function (physical, role, cognitive, social, and emotional), multi-term symptoms (fatigue and pain), and single items (appetite loss and insomnia). Occurrence of symptom, impacts, and QoL/function improvement based on best overall response will be compared between treatment groups using a logistic regression model adjusting for the same factors as the primary endpoint (disease status and primary tumor location). The odds ratio, p-value, and 95% CI will be presented graphically on a forest plot.

Finally, summaries of absolute and unadjusted change from baseline values of each EORTC QLQ-C30 scale/item will be reported by visit for each treatment group. Graphical presentations may also be produced as appropriate.

Additional supportive/exploratory analyses may be conducted.

### EORTC QLQ-BIL21

The primary assessment of TTD, as described for the EORTC QLQ-C30, will be evaluated for single-item abdominal pain (item 42), pruritus (item 36), and jaundice (item 35) symptoms of the EORTC QLQ-BIL21.

For TTD in abdominal pain, pruritus, and jaundice, single items of the EORTC QLQ-BIL21 will be presented using a Kaplan-Meier plot as well as the HR together with the corresponding 95% CI and p-values. Summaries of the number and percentage of patients experiencing a clinically meaningful deterioration or death and the median TTD will also be provided for each treatment group.

Additionally, comparing mean change from baseline using the MMRM as described for the EORTC QLQ-C30 will be repeated for abdominal pain, pruritus, and jaundice symptoms of the EORTC QLQ-BIL21. All assumptions and outputs as described for the EORTC QLQ-C30 are applicable.

Summary tables of visit responses for each EORTC QLQ-BIL21 scale/item score and for each visit (improvement, deterioration, and no change) will be presented by treatment group. In addition, summary tables of best overall response will be provided for abdominal pain, pruritus, and jaundice symptoms by treatment group. Occurrence of improvement based on best overall response will be compared between treatment groups using a logistic regression model. The odds ratio, p-value, and 95% CI will be presented graphically on a forest plot.

As described for the EORTC QLQ-C30, summaries of absolute and unadjusted change from baseline values of each EORTC QLQ-BIL21 scale/item will be reported by visit for each treatment group. Graphical presentations may also be produced as appropriate. This descriptive analysis includes item 49 of the EORTC QLQ-BIL21 (“To what extent have you been troubled with side-effects from your treatment?”) assessing patient's global impression of treatment tolerability, which will complement exploratory findings of the PRO-CTCAE assessment.

### PRO-CTCAE

PRO-CTCAE data will be presented using summaries and descriptive statistics based on the FAS. EORTC QLQ-BIL21 item 49 (“To what extent have you been troubled with side-effects from your treatment?”) descriptive statistics will complement PRO-CTCAE findings. Refer to the section above.

### PGIS

Responses on the PGIS will be summarized descriptively as number of patients and corresponding percentages for each category in the questionnaire at each visit by treatment group.

### EQ-5D-5L

Descriptive statistics will be calculated for each scheduled visit/timepoint in the study, for each study arm, and as a total. This will report the number of patients, the number of EQ-5D questionnaires completed at each visit, and the number and proportion responding to each dimension of the EQ-5D-5L. Additionally, summary statistics (e.g., n, mean, median, SD, min, and max) will be reported for the EQ-5D index score and the EQ-VAS score, as well as the change from baseline for the EQ-5D index score and the EQ-VAS score.

Graphical plots of the mean EQ-5D index score and EQ-VAS score, including change from baseline, and associated 95% CI by scheduled visits/timepoints in the study may be produced. To support submissions to payers, additional analyses may be undertaken, and these will be outlined in a separate Payer Analysis Plan.

### Example 2: Global Phase 3 Clinical Trial of the Efficacy of Durvalumab in Combination with Chemotherapy in Patients with Biliary Tract Cancer (BTC)

### Patients

Adults aged ≥18 years with histologically confirmed unresectable, locally advanced, or metastatic adenocarcinoma of the biliary tract, including intrahepatic or extrahepatic cholangiocarcinoma and gallbladder carcinoma, were eligible for inclusion. Eligible patients included those with previously untreated disease that was unresectable or metastatic at initial diagnosis and those who developed recurrent disease >6 months after surgery with curative intent and >6 months after the completion of adjuvant therapy. Other inclusion criteria included an Eastern Cooperative Oncology Group (ECOG) performance status of 0 or 1, ≥1 measurable lesion per Response Evaluation Criteria in Solid Tumors (RECIST) v1.1, and no prior exposure to immune-meditated therapy. Patients were excluded if they had ampullary carcinoma, active or prior documented autoimmune or inflammatory disorders, or known allergy or hypersensitivity to any study treatment.

### Trial Design and Interventions

TOPAZ-1 was a phase 3, randomized, double-blind, placebo-controlled, global study. Patients were randomly assigned in a 1:1 ratio to receive durvalumab in combination with gemcitabine and cisplatin or placebo in combination with gemcitabine and cisplatin. Randomization was stratified by disease status (initially unresectable versus recurrent) and primary tumor location (intrahepatic cholangiocarcinoma versus extrahepatic cholangiocarcinoma versus gallbladder cancer).

Durvalumab or placebo combined with gemcitabine and cisplatin was administered intravenously on a 21-day cycle for up to 8 cycles. Durvalumab 1500 mg or placebo was administered on day 1 of each cycle, in combination with gemcitabine 1000 mg/m² and cisplatin 25 mg/m², which were administered on days 1 and 8 of each cycle. After completion of gemcitabine and cisplatin administration, durvalumab 1500 mg or placebo monotherapy was administered once every 4 weeks until clinical or imaging (per RECIST v1.1) disease progression or until unacceptable toxicity, withdrawal of consent, or any other discontinuation criteria were met. Patients who were clinically stable at initial disease progression could continue to receive study treatment at the discretion of the investigator and patient.

### Assessments

Tumor assessments were performed according to RECIST v1.1 using images obtained by computed tomography or magnetic resonance imaging of the chest, abdomen, and pelvis, and evaluated per investigator assessment. Adverse events were reported from the time of informed consent through 90 days after the last dose of study treatment; the causal relationship between reported adverse events and study treatment was investigator-assessed. Other safety assessments included physical examinations, laboratory findings, ECOG performance status, electrocardiograms, and vital signs. Health-related quality of life was assessed using the European Organisation for Research and Treatment of Cancer (EORTC)-30-Item Core Quality of Life Questionnaire and the EORTC-21-Item Cholangiocarcinoma and Gallbladder Cancer Quality of Life Questionnaire. Patient-reported treatment tolerability was assessed using the Patient-Reported Outcomes-Common Terminology Criteria for Adverse Events.

Baseline tumor assessments were performed using images acquired≤28 days before randomization. During the study period, tumor assessments were performed every 6 weeks (±1 week) for the first 24 weeks after randomization, and then every 8 weeks (±1 week) thereafter, until radiological disease progression, followed by ≥1 additional follow-up scan. Survival assessments were performed every 2 months following treatment discontinuation. Patients on treatment or in survival follow-up were contacted following the data cutoff for the interim analysis to provide complete survival data. Newly acquired tumor biopsy samples or archival samples taken≤3 years prior to screening were obtained by an image-guided core needle or by excision from all screened patients. Tissue samples were retrospectively evaluated for programmed cell death ligand 1 (PD-L1) expression by the Ventana PD-L1 (SP263) immunohistochemistry assay in all randomized patients using the tumor area positivity (TAP) score method. “TAP” is the proportion of tumor and/or immune cells with PD-L1 staining at any intensity and can be used interchangeably with “TIP.” A TAP score of ≥1% was defined as PD-L1 staining of any intensity in tumor cell membranes and/or tumor-associated immune cells covering ≥1% of the tumor area. A TAP score of <1% was defined as PD-L1 staining of any intensity in tumor cell membranes and/or tumor-associated immune cells covering <1% of tumor area. The impact of PD-L1 expression on response and survival outcomes of patients in the durvalumab and placebo treatment groups was evaluated. MSI status was determined by Foundation Medicine, Inc. (as described in U.S. Food and Drug Administration, 2017) using data generated with the FoundationOne assay to analyze tumor tissue samples. In brief, principal components analysis was used to analyze length variability of the 95 intronic homopolymer repeat loci and samples were assigned a qualitative status of MSI-high or MSI-stable. Samples with low coverage (<250× median) were assigned a status of MSI-unknown.

### Objectives and Endpoints

The primary objective was to assess overall survival, defined as the time between randomization and death due to any cause, in the durvalumab versus the placebo group. Secondary endpoints included progression-free survival, objective response rate, duration of response, and disease control rate and efficacy by PD-L1 expression, according to RECIST v1.1 using investigator assessments, in the durvalumab versus placebo groups. Progression-free survival is defined as the time from date of randomization until the date of RECIST v1.1 defined imaging disease progression or death. Safety and tolerability of the durvalumab and placebo groups were assessed. Adverse events were graded according to National Cancer Institute common terminology criteria for adverse events, version 5.0.

### Statistical Analysis

Efficacy objectives were evaluated in the full analysis set, which included all randomized patients. The primary analysis of overall survival tested for superiority of the durvalumab regimen relative to placebo and was analyzed using a log-rank test stratified by disease status (initially unresectable versus recurrent) and primary tumor location (intrahepatic cholangiocarcinoma versus extrahepatic cholangiocarcinoma versus gallbladder cancer). This interim analysis was planned for when approximately 397 of the expected 496 deaths at final analysis had occurred, providing an approximate 75% power to detect a significant difference in overall survival with a two-sided significance level of 0.0238 according to the Lan-DeMets approximation of O'Brien Fleming alpha spending function boundaries. By the data cutoff date, 424 deaths had occurred, and the actual two-sided significance level was 0.03. As a statistically significant improvement in overall survival in the durvalumab arm compared with the placebo arm was observed at the planned interim analysis, the key secondary endpoint of progression-free survival was formally evaluated at this interim analysis. For overall survival and progression-free survival, the effect of durvalumab versus placebo was estimated by the hazard ratio with a 95% confidence interval and two-sided P value. Progression-free survival in the durvalumab versus placebo treatment groups was tested using a log-rank test, with a two-sided significance level of 0.0481 that was derived based on the alpha spending function approximating Pocock boundaries. No multiplicity adjustments for the other secondary and exploratory end points were defined. Therefore, only point estimates and 95% confidence intervals are provided. The confidence intervals for endpoints other than overall survival and progression-free survival have not been adjusted for multiple comparisons and should not be used to infer definitive treatment effects.

Objective response rate was compared between the durvalumab and the placebo treatment groups using a Cochran-Mantel-Haenszel test stratified by disease status (initially unresectable versus recurrent) and primary tumor location (intrahepatic cholangiocarcinoma versus extrahepatic cholangiocarcinoma versus gallbladder cancer). Safety was assessed in all patients who received at least one dose of the study treatment, and data were summarized with descriptive statistics. All confidence intervals reported have not been adjusted for multiplicity and cannot be used to infer effects.

The results of this planned interim analysis were reviewed by an independent data monitoring committee that concluded that the data met the prespecified criteria for a statistically significant difference in the primary objective, overall survival, per the statistical analysis plan with acceptable safety. Since the trial reached statistical significance for the primary objective on the basis of this prespecified interim analysis, the sponsor was unblinded, and the results presented herein are to be considered the final, formal statistical analysis for overall survival. The TOPAZ-1 study is ongoing, allowing for further, exploratory follow-up overall survival analyses.

### Results

### Patients and Treatment

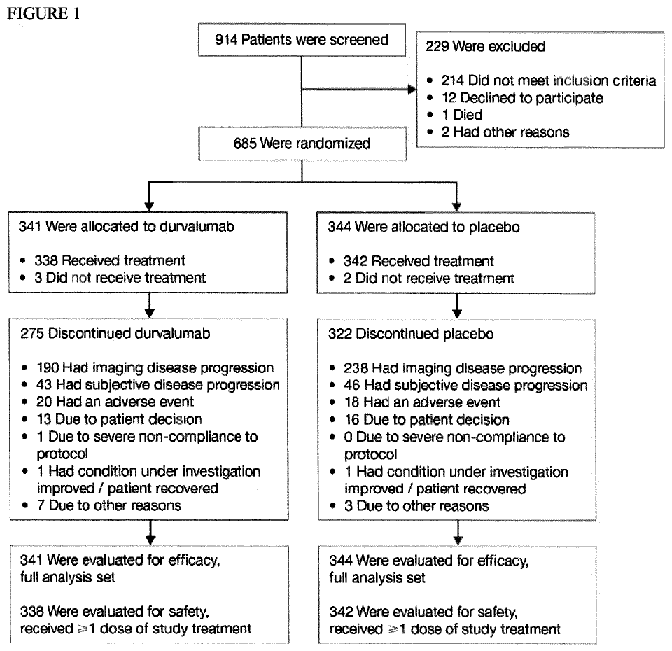

During the study, 914 patients were enrolled at 105 sites in 17 countries. In total, 685 patients were randomized, 341 to the durvalumab group and 344 to the placebo group, and 338 and 342 patients, respectively, received treatment (FIG. 1). Patient demographics and disease characteristics were generally balanced between the treatment groups (Tables 8-10). The representativeness of the trial participants in TOPAZ-1 is described in Table 11. As of the data cutoff date, the median (95% CI) duration of follow-up was 16.8 (14.8-17.7) months in the durvalumab group and 15.9 (14.9-16.9) months in the placebo group. Additionally, 275 (81.4%) patients in the durvalumab group discontinued durvalumab and 322 (94.2%) patients in the placebo group discontinued placebo. Of the full analysis set, the number of patients who received≥1 regimen of subsequent anticancer therapy post-discontinuation was 145 (42.5%) in the durvalumab group and 170 (49.4%) in the placebo group (Table 12).

TABLE 8
Patient Demographics and Baseline Characteristics in the Full Analysis Set.

| | Durvalumab | | | | | |
|---|---|---|---|---|---|---|
| | plus | | Placebo plus | | | |
| | Gemcitabine | | Gemcitabine | | | |
| | and Cisplatin | | and Cisplatin | | Total | |
| Parameter | (n = 341) | | (n = 344) | | (n = 685) | |
| Median age (range) - yr | 64 | (20-84) | 64 | (31-85) | 64 | (20-85) |
| Female sex - no. (%) | 172 | (50.4) | 168 | (48.8) | 340 | (49.6) |
| Race - no. (%) | | | | | | |
| Asian | 185 | (54.3) | 201 | (58.4) | 386 | (56.4) |
| Region - no. (%) | | | | | | |
| Asia | 178 | (52.2) | 196 | (57.0) | 374 | (54.6) |
| ROW | 163 | (47.8) | 148 | (43.0) | 311 | (45.4) |
| ECOG performance status - no. (%) | | | | | | |
| 0 | 173 | (50.7) | 163 | (47.4) | 336 | (49.1) |
| Primary tumor type - no. (%) | | | | | | |
| Intrahepatic cholangiocarcinoma | 190 | (55.7) | 193 | (56.1) | 383 | (55.9) |
| Extrahepatic cholangiocarcinoma | 66 | (19.4) | 65 | (18.9) | 131 | (19.1) |
| Gallbladder | 85 | (24.9) | 86 | (25.0) | 171 | (25.0) |
| Disease status - no. (%) | | | | | | |
| Initially unresectable | 274 | (80.4) | 279 | (81.1) | 553 | (80.7) |
| Recurrent | 67 | (19.6) | 64 | (18.6) | 131 | (19.1) |
| Disease classification - no. (%) | | | | | | |
| Locally advanced* | 38 | (11.1) | 57 | (16.6) | 95 | (13.9) |
| Metastatic | 303 | (88.9) | 286 | (83.1) | 589 | (86.0) |
| MSI status - no. (%) | | | | | | |
| High | 3 | (0.9) | 2 | (0.6) | 5 | (0.7) |
| Stable | 160 | (46.9) | 168 | (48.8) | 328 | (47.9) |
| Missing† | 178 | (52.2) | 174 | (50.6) | 352 | (51.4) |
| Virology status - no. (%) | | | | | | |
| No viral hepatitis | 187 | (54.8) | 174 | (50.6) | 361 | (52.7) |
| Any viral hepatitis B | 69 | (20.2) | 81 | (23.5) | 150 | (21.9) |
| Active viral hepatitis B | 8 | (2.3) | 14 | (4.1) | 22 | (3.2) |
| Prior hepatitis C | 8 | (2.3) | 10 | (2.9) | 18 | (2.6) |
| Missing | 82 | (24.0) | 83 | (24.1) | 165 | (24.1) |
| PD-L1 expression - no. (%) | | | | | | |
| TAP ≥1% | 197 | (57.8) | 205 | (59.6) | 402 | (58.7) |
| TAP <1% | 103 | (30.2) | 103 | (29.9) | 206 | (30.1) |
| Missing | 41 | (12.0) | 36 | (10.5) | 77 | (11.2) |

*Patient has only locally advanced sites of disease.
†MSI status missing includes MSI-unknown and not tested.
ECOG = Eastern Cooperative Oncology Group, MSI = microsatellite instability, PD-L1 = programmed cell death ligand 1, and TAP = tumor area positivity (proportion of tumor and/or immune cells with PD-L1 staining at any intensity).

TABLE 9
Patient Demographics and Baseline Characteristics in the Full Analysis Set.

| | Durvalumab | | | | | |
|---|---|---|---|---|---|---|
| | plus | | Placebo plus | | | |
| | Gemcitabine | | Gemcitabine | | | |
| | and Cisplatin | | and Cisplatin | | Total | |
| Parameter | (n = 341) | | (n = 344) | | (n = 685) | |
| Median age (range) - yr | 64 | (20-84) | 64 | (31-85) | 64 | (20-85) |
| Female sex - no. (%) | 172 | (50.4) | 168 | (48.8) | 340 | (49.6) |
| Race - no. (%) | | | | | | |
| Asian | 185 | (54.3) | 201 | (58.4) | 386 | (56.4) |
| White | 131 | (38.4) | 124 | (36.0) | 255 | (37.2) |
| Black or African American | 8 | (2.3) | 6 | (1.7) | 14 | (2.0) |
| American Indian or Alaska Native | 0 | | 1 | (0.3) | 1 | (0.1) |
| Other | 17 | (5.0) | 12 | (3.5) | 29 | (4.2) |
| Region - no. (%) | | | | | | |
| Asia | 178 | (52.2) | 196 | (57.0) | 374 | (54.6) |
| Europe | 108 | (31.7) | 107 | (31.1) | 215 | (31.4) |
| North America | 37 | (10.9) | 28 | (8.1) | 65 | (9.5) |
| South America | 18 | (5.3) | 13 | (3.8) | 31 | (4.5) |
| ECOG performance status - no. (%) | | | | | | |
| 0 | 173 | (50.7) | 163 | (47.4) | 336 | (49.1) |
| 1 | 168 | (49.3) | 181 | (52.6) | 349 | (50.9) |
| Primary tumor type - no. (%) | | | | | | |
| Intrahepatic cholangiocarcinoma | 190 | (55.7) | 193 | (56.1) | 383 | (55.9) |
| Extrahepatic cholangiocarcinoma | 66 | (19.4) | 65 | (18.9) | 131 | (19.1) |
| Gallbladder | 85 | (24.9) | 86 | (25.0) | 171 | (25.0) |
| Disease status - no. (%) | | | | | | |
| Initially unresectable | 274 | (80.4) | 279 | (81.1) | 553 | (80.7) |
| Recurrent | 67 | (19.6) | 64 | (18.6) | 131 | (19.1) |
| Missing | 0 | | 1 | (0.3) | 1 | (0.1) |
| Disease classification - no. (%) | | | | | | |
| Locally advanced* | 38 | (11.1) | 57 | (16.6) | 95 | (13.9) |
| Metastatic | 303 | (88.9) | 286 | (83.1) | 589 | (86.0) |
| Missing | 0 | | 1 | (0.3) | 1 | (0.1) |
| MSI status - no. (%) | | | | | | |
| High | 3 | (0.9) | 2 | (0.6) | 5 | (0.7) |
| Stable | 160 | (46.9) | 168 | (48.8) | 328 | (47.9) |
| Missing† | 178 | (52.2) | 174 | (50.6) | 352 | (51.4) |
| Virology status - no. (%) | | | | | | |
| No viral hepatitis | 187 | (54.8) | 174 | (50.6) | 361 | (52.7) |
| Any viral hepatitis B | 69 | (20.2) | 81 | (23.5) | 150 | (21.9) |
| Active viral hepatitis B | 8 | (2.3) | 14 | (4.1) | 22 | (3.2) |
| Prior hepatitis C | 8 | (2.3) | 10 | (2.9) | 18 | (2.6) |
| Missing | 82 | (24.0) | 83 | (24.1) | 165 | (24.1) |
| PD-L1 expression - no. (%) | | | | | | |
| TAP ≥1% | 197 | (57.8) | 205 | (59.6) | 402 | (58.7) |
| TAP <1% | 103 | (30.2) | 103 | (29.9) | 206 | (30.1) |
| Missing | 41 | (12.0) | 36 | (10.5) | 77 | (11.2) |

*Patient has only locally advanced sites of disease.
†MSI status missing includes MSI-unknown and not tested.
ECOG = Eastern Cooperative Oncology Group, MSI = microsatellite instability, PD-L1 = programmed cell death ligand 1, and TAP = tumor area positivity (proportion of tumor and/or immune cells with PD-L1 staining at any intensity).

TABLE 10
Extent of Disease at Baseline in the Full Analysis Set.

| | Durvalumab | | |
|---|---|---|---|
| | plus | Placebo plus | |
| | Gemcitabine | Gemcitabine | |
| | and Cisplatin | and Cisplatin | Total |
| Site of disease | (n = 341) | (n = 344) | (n = 685) |

Locally advanced sites of disease - no. (%)

| | | | | | | |
|---|---|---|---|---|---|---|
| Liver | 94 | (27.6) | 113 | (32.8) | 207 | (30.2) |
| Regional lymph nodes | 71 | (20.8) | 77 | (22.4) | 148 | (21.6) |
| Gallbladder | 47 | (13.8) | 47 | (13.7) | 94 | (13.7) |
| Hepatic (including gallbladder) | 41 | (12.0) | 21 | (6.1) | 62 | (9.1) |
| Metastatic sites of disease - no. (%) | | | | | | |
| Liver | 160 | (46.9) | 133 | (38.7) | 293 | (42.8) |
| Distant lymph nodes | 134 | (39.3) | 135 | (39.2) | 269 | (39.3) |
| Regional lymph nodes | 89 | (26.1) | 75 | (21.8) | 164 | (23.9) |
| Peritoneum | 88 | (25.8) | 79 | (23.0) | 167 | (24.4) |
| Respiratory | 88 | (25.8) | 76 | (22.1) | 164 | (23.9) |
| Hepatic (including gallbladder) | 33 | (9.7) | 38 | (11.0) | 71 | (10.4) |
| Bone and locomotor | 30 | (8.8) | 20 | (5.8) | 50 | (7.3) |
| Ascites | 26 | (7.6) | 26 | (7.6) | 52 | (7.6) |

Data include sites of disease with a frequency of >5% in either treatment arm.

TABLE 11
Representativeness of Trial Participants in TOPAZ-1.

| Category | Details |
|---|---|
| Disease, problem, or condition | Biliary tract cancer |
| under investigation | |

Special considerations related to:

| | |
|---|---|
| Sex and gender | Globally, incidence rates of extrahepatic |
| | cholangiocarcinoma and intrahepatic |
| | cholangiocarcinoma tend to be higher in males |
| | than in females, while gallbladder cancer tends |
| | to be more common in females than in males |
| | (Wang et al., 2020; Van Dyke et al., 2019; Kim |
| | et al., 2019). In the United States, male to |
| | female incidence rate ratios of 0.8 for |
| | intrahepatic cholangiocarcinoma, 0.7 for |
| | extrahepatic cholangiocarcinoma, and 1.7 for |
| | gallbladder cancer have been reported (Van |
| | Dyke et al., 2019). |
| Age | The incidence of biliary tract cancer increases |
| | with increasing age, with peak incidence |
| | observed among individuals in their 70s and 80s |
| | (Van Dyke et al., 2019; Kim et al., 2019). |
| Race and ethnic group | In the United States, the incidence of biliary tract |
| | cancer is generally higher in Hispanic and Black |
| | individuals than non-Hispanic White individuals |
| | (Van Dyke et al., 2019). A higher incidence rate |
| | of biliary tract cancers has also been reported for |
| | Asian Americans compared with the general |
| | United States population (Wang et al., 2020). |
| Geography | The incidence of biliary tract cancer varies |
| | worldwide (Bridgewater et al., 2016). The |
| | highest incidences of cholangiocarcinoma have |
| | generally been reported in Asian countries, and |
| | rates >6 per 100,000 individuals have been |
| | reported for China, Thailand, and South Korea |
| | (id.). Although the incidence of biliary tract |
| | cancer is generally low in Europe and North |
| | America, the incidence of intrahepatic |
| | cholangiocarcinoma has been increasing in the |
| | last three decades (id.). |
| Other considerations | Survival rates for patients with biliary tract |
| | cancer vary substantially worldwide and are |
| | higher in women than in men for some countries |
| | (Torre et al., 2018). For patients treated with |
| | standard of care for advanced disease, |
| | gemcitabine and cisplatin, survival rates are |
| | comparable between Asian and non-Asian |
| | countries (Valle et al., 2010; Okusaka et al., |
| | 2010). |
| Overall | Patients enrolled in the TOPAZ-1 study self- |
| representativeness | reported their sex as male or female during the |
| of this trial | trial screening. Overall, males and females were |
| | equally represented in the full analysis set, |
| | reflecting trends seen in the general patient |
| | population for BTC. The median age of 64.0 |
| | years was younger than the peak ages of |
| | incidence reported in the literature. However, |
| | this is consistent with median ages in clinical |
| | trials for advanced biliary tract cancer (Valle et |
| | al., 2010; Okusaka et al., 2010). Consistent with |
| | reported incidences of biliary tract cancers by |
| | geographic region (Bridgewater et al., 2016), a |
| | substantial proportion (54.6%) of the full |
| | analysis set was from Asia, and 56.4% of |
| | patients self-reported as Asian. The second most |
| | reported race was White (37.2%). Two percent |
| | of patients were Black or African American, and |
| | 6.7% of patients were Hispanic or Latino. |
| | Therefore, the patient population in TOPAZ-1 is |
| | an accurate representation of the global |
| | incidence of advanced biliary tract cancer (id.). |

TABLE 12
Subsequent Anticancer Therapies in All Treatment
Groups in the Full Analysis Set.

| | Durvalumab plus Gemcitabine | | Placebo plus Gemcitabine | |
|---|---|---|---|---|
| | and Cisplatin | | and Cisplatin | |
| | (n = 341) | | (n = 344) | |
| Any subsequent anticancer | 145 | (42.5) | 170 | (49.4) |
| therapy - no. (%) | | | | |
| Immunotherapy | 3 | (0.9) | 16 | (4.7) |
| Targeted therapy | 12 | (3.5) | 16 | (4.7) |
| Cytotoxic chemotherapy | 137 | (40.2) | 156 | (45.3) |
| Taxane chemotherapy | 5 | (1.5) | 9 | (2.6) |
| Antiangiogenic therapy | 1 | (0.3) | 1 | (0.3) |
| Other | 15 | (4.4) | 28 | (8.1) |

Data include all patients who discontinued study treatment and received at least one dose of anticancer therapy.

### Primary Objective

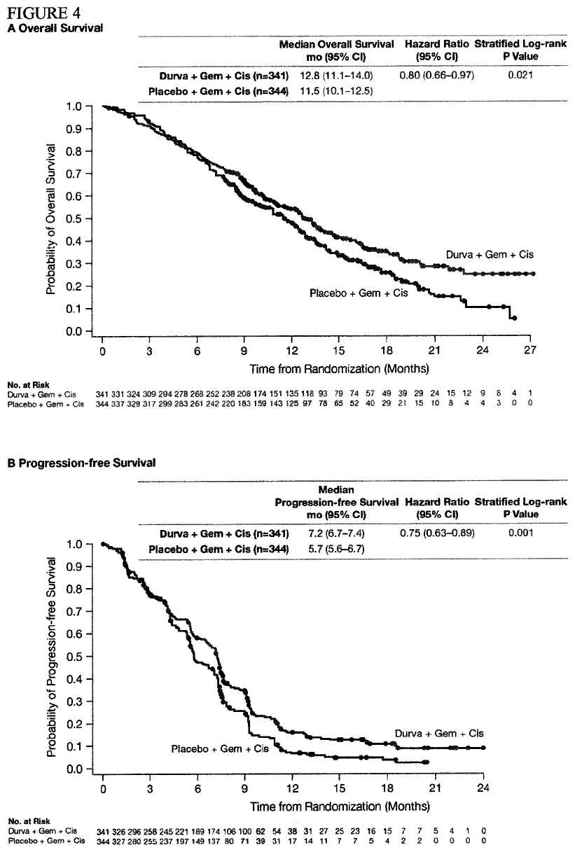

As of data cutoff, 198 (58.1%) patients in the durvalumab group and 226 (65.7%) patients in the placebo group had died. Overall survival was significantly longer with durvalumab versus placebo (hazard ratio, 0.80; 95% CI, 0.66-0.97; P=0.021). Median overall survival (95% CI) was 12.8 (11.1-14.0) months in the durvalumab group and 11.5 (10.1-12.5) months in the placebo treatment group (FIG. 4A).

The estimated overall survival rates (95% CI) were 54.1% (48.4-59.4) for durvalumab and 48.0% (42.4-53.4) for placebo at 12 months, 35.1% (29.1-41.2) for durvalumab and 25.6% (19.9-31.7) for placebo at 18 months, and 24.9% (17.9-32.5) for durvalumab and 10.4% (4.7-18.8) for placebo at 24 months.

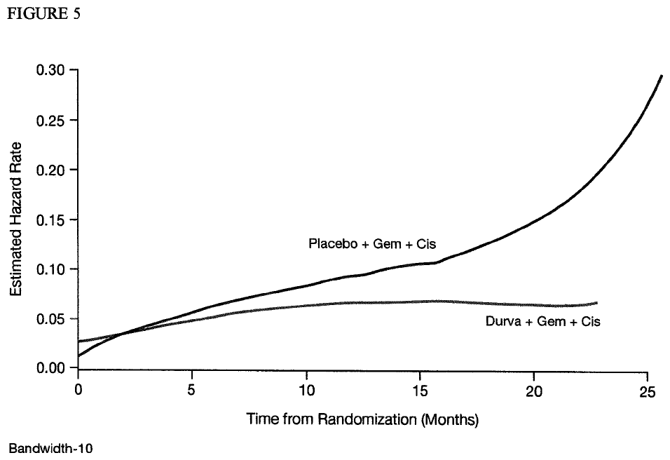

The overall survival Kaplan-Meier curve separated at approximately 6 months of treatment, after which there was a clear and sustained separation of the survival curves in favor of the durvalumab group. The overall survival hazard ratio (95% CI) was 0.91 (0.66-1.26) up to 6 months and 0.74 (0.58-0.94) after 6 months. Furthermore, a kernel-smoothed estimate of the hazard function and the associated log-log (event times) versus log (time) plot confirmed a departure from the assumption of the proportional hazards (FIG. 5).

### Secondary Objectives

Median (95% CI) progression-free survival was 7.2 (6.7-7.4) months with durvalumab and 5.7 (5.6-6.7) months with placebo (FIG. 4B). The hazard ratio for progression-free survival was 0.75 (95% CI, 0.63-0.89; P=0.001). The investigator-assessed confirmed objective response rate (sum of the rate of complete responses and partial responses in patients with measurable disease) was 26.7% in the durvalumab group (n=341) and 18.7% in the placebo group (n=343) (odds ratio, 1.60; 95% CI, 1.11-2.31). The number of patients achieving a confirmed complete response was 7 (2.1%) with durvalumab and 2 (0.6%) with placebo, and the number of patients achieving a confirmed partial response was 84 (24.6%) with durvalumab and 62 (18.1%) with placebo. The percentage of patients with continued response for ≥9 months was 32.6% with durvalumab and 25.3% with placebo. The percentage of patients with continued response for ≥12 months was 26.1% with durvalumab and 15.0% with placebo. Tumor responses are summarized in Table 13.

TABLE 13
Tumor Response in the Full Analysis Set.

| | Durvalumab plus | | Placebo plus | |
|---|---|---|---|---|
| | Gemcitabine and | | Gemcitabine and | |
| | Cisplatin | | Cisplatin | |
| Parameter | (n = 341) | | (n = 343) | |
| Objective response rate - no. (%)* | 91 | (26.7) | 64 | (18.7) |
| Complete response | 7 | (2.1) | 2 | (0.6) |
| Partial response | 84 | (24.6) | 62 | (18.1) |
| Disease control rate - no. (%)† | 291 | (85.3) | 284 | (82.6) |
| Median duration of response (IQR) - mo‡ | 6.4 | (4.6-17.2) | 6.2 | (3.8-9.0) |

Percentage of patients with continued response - %

| ≥3 months | 88.9 | | 89.0 | |
|---|---|---|---|---|
| ≥6 months | 59.3 | | 54.2 | |
| ≥9 months | 32.6 | | 25.3 | |
| ≥12 months | 26.1 | | 15.0 | |
| Median time to response (IQR) - mo§ | 1.6 | (1.3-3.0) | 2.7 | (1.4-4.1) |
| Median time to response (IQR) - mo§ | 1.6 | (1.3-3.0) | 2.7 | (1.4-4.1) |

*By investigator assessment using RECIST v1.1. Analysis of objective response rate was based on patients in the final analysis set who had measurable disease at baseline. There was one patient who did not have measurable disease at baseline in the placebo group.
†The rate of best objective response of complete response, partial response, and stable disease. Analysis of disease control rate was based on all patients in the full analysis set (n = 341 for durvalumab and n = 344 for placebo).
‡Time from the first documentation of a response until the date of progression, death, or the last evaluable RECIST assessment. Analysis of duration of response was based on patients in the full analysis set who had an objective response and measurable disease at baseline (n = 91 for durvalumab and n = 64 for placebo).
§Time from the date of randomization until the date of first documented response. Analysis of time to response was based on patients in the full analysis set who had an objective response and measurable disease at baseline (n = 91 for durvalumab and n = 64 for placebo).
IQR = interquartile range, and RECIST v1.1 = Response Evaluation Criteria in Solid Tumors, version 1.1.

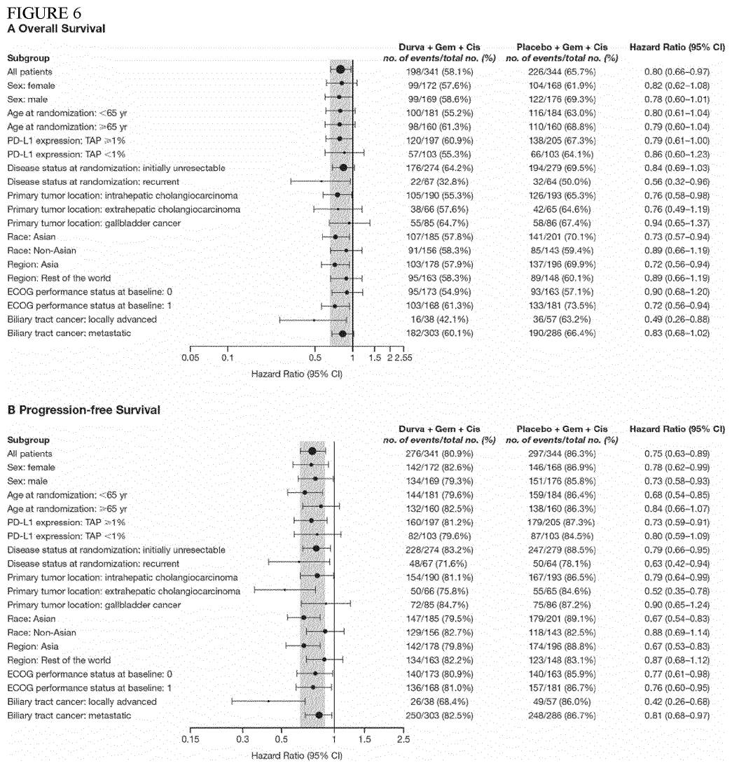

The overall and progression-free survival benefits observed with durvalumab in combination with gemcitabine and cisplatin were generally consistent across the clinically relevant subgroups analyzed (FIGS. 6A and 6B). In patients with a PD-L1 tumor area positivity (TAP) score of >1% (>1% of tumor area occupied by tumor and/or immune cells with PD-L1 staining at any intensity), the hazard ratio for overall survival with durvalumab versus placebo was 0.79 (95% CI, 0.61-1.00). In patients with a TAP score <1%, the hazard ratio for overall survival with durvalumab versus placebo was 0.86 (95% CI, 0.60-1.23).

### Safety

The safety analysis set included 680 patients who received ≥1 dose of durvalumab (n=338) or placebo (n=342). The median (range) duration of study treatment was 7.3 (0.1-24.5) months for durvalumab and 5.8 (0.2-21.5) months for placebo (Table 14).

TABLE 14
Treatment Exposure in the Safety Analysis Set.

| | Durvalumab plus | | Placebo plus | |
|---|---|---|---|---|
| | Gemcitabine and | | Gemcitabine and | |
| | Cisplatin (n = 338) | | Cisplatin (n = 342) | |
| Median duration of exposure | 7.3 | (0.1-24.5) | 5.8 | (0.2-21.5) |
| of durvalumab/placebo, | | | | |
| months (range) | | | | |
| Median duration of exposure | 5.2 | (0.1-8.3) | 5.0 | (0.2-8.6) |
| of gemcitabine, months | | | | |
| (range) | | | | |
| Median duration of exposure | 5.1 | (0.1-8.3) | 4.9 | (0.2-8.5) |
| of cisplatin, months (range) | | | | |
| Median number of | 10.0 | (1-29) | 8.0 | (1-26) |
| durvalumab/placebo cycles | | | | |
| (range) | | | | |
| Median number of | 8.0 | (0-8) | 8.0 | (1-8) |
| gemcitabine cycles (range) | | | | |
| Median number of cisplatin | 8.0 | (0-8) | 7.5 | (1-8) |
| cycles (range) | | | | |
| Median relative dose | 100 | (93.8-100) | 100 | (95.0-100) |
| intensity of durvalumab/ | | | | |
| placebo (IQR) | | | | |
| Median relative dose | 93.8 | (82.5-100) | 93.8 | (82.2-100) |
| intensity of gemcitabine | | | | |
| (IQR) | | | | |
| Median relative dose | 93.8 | (83.3-100) | 93.8 | (81.3-100) |

intensity of cisplatin (IQR)

IQR = interquartile range.

In the durvalumab group, the median (interquartile range) relative dose intensity of durvalumab, gemcitabine, and cisplatin were 100 (93.8-100), 93.8 (82.5-100), and 93.8 (83.3-100), respectively. In the placebo group, the median (interquartile range) relative dose intensity of placebo, gemcitabine and cisplatin were 100 (95.0-100), 93.8 (82.2-100), and 93.8 (81.3-100), respectively.

Any grade adverse events occurred in 336 (99.4%) patients in the durvalumab group and 338 (98.8%) patients in the placebo group (Table 15). Grade 3/4 adverse events occurred in 256 (75.7%) patients in the durvalumab group and 266 (77.8%) patients in the placebo group (Table 15). The rate of discontinuation of any treatment component due to possibly treatment-related adverse events was 8.9% in the durvalumab group and 11.4% in the placebo group (Table 15). The number of deaths due to adverse events was 12 (3.6%) in the durvalumab group and 14 (4.1%) in the placebo group. Deaths due to treatment-related adverse events occurred in 2 (0.6%) patients in the durvalumab group and 1 (0.3%) patient in the placebo group (Table 15). The most common adverse events were anemia (48.2%), nausea (40.2%), constipation (32.0%), and neutropenia (31.7%) in the durvalumab group, and anemia (44.7%), nausea (34.2%), and decreased neutrophil count (31.0%) in the placebo group (Table 16). Grade 3 or 4 treatment-related adverse events that occurred in ≥2% of patients in the durvalumab and placebo groups are listed in Table 17. The rate of immune-mediated adverse events was 12.7% with durvalumab and 4.7% with placebo. Grade 3 or 4 immune-mediated adverse events occurred in 2.4% of patients in the durvalumab group and 1.5% of in the placebo group (Table 18).

TABLE 15
Summary of Safety Data in the Safety Analysis Set.

| | Durvalumab plus | | Placebo plus | |
|---|---|---|---|---|
| | Gemcitabine | | Gemcitabine | |
| | and Cisplatin | | and Cisplatin | |
| | (n = 338) | | (n = 342) | |
| Adverse events - no. (%) | | | | |
| Adverse event of any grade | 336 | (99.4) | 338 | (98.8) |
| Serious adverse event | 160 | (47.3) | 149 | (43.6) |
| Adverse event of grade 3 or 4 | 256 | (75.7) | 266 | (77.8) |
| Adverse event leading to | 44 | (13.0) | 52 | (15.2) |
| discontinuation | | | | |
| Adverse event leading to death | 12 | (3.6) | 14 | (4.1) |

Treatment-related adverse events - no. (%)

| | | | | |
|---|---|---|---|---|
| Adverse event of any grade | 314 | (92.9) | 308 | (90.1) |
| Serious adverse event | 53 | (15.7) | 59 | (17.3) |
| Adverse event of grade 3 or 4 | 212 | (62.7) | 222 | (64.9) |
| Adverse event leading to | 30 | (8.9) | 39 | (11.4) |
| discontinuation of any study | | | | |
| treatment | | | | |
| Adverse event leading to death* | 2 | (0.6) | 1 | (0.3) |

*Treatment-related adverse events leading to death were ischemic stroke and hepatic failure in the durvalumab treatment group and polymyositis in the placebo treatment group.

TABLE 16
Adverse Events of Any Causality of Any Grade with an Incidence of ≥10% in Either
Treatment Group or of Grade 3 or 4 with an incidence of ≥2% in Either
Treatment Group in the Safety Analysis Set.

| | Durvalumab plus | | | | Placebo plus Gemcitabine | | | |
|---|---|---|---|---|---|---|---|---|
| | Gemcitabine and Cisplatin | | | | and Cisplatin | | | |
| | (n = 338) | | | | (n = 342) | | | |
| Adverse Events - no. (%) | Any Grade | | Grade 3/4 | | Any Grade | | Grade 3/4 | |
| Anemia | 163 | (48.2) | 80 | (23.7) | 153 | (44.7) | 77 | (22.5) |
| Nausea | 136 | (40.2) | 5 | (1.5) | 117 | (34.2) | 6 | (1.8) |
| Constipation | 108 | (32.0) | 2 | (0.6) | 99 | (28.9) | 1 | (0.3) |
| Neutropenia | 107 | (31.7) | 68 | (20.1) | 102 | (29.8) | 72 | (21.1) |
| Neutrophil count decreased | 91 | (26.9) | 71 | (21.0) | 106 | (31.0) | 88 | (25.7) |
| Fatigue | 91 | (26.9) | 11 | (3.3) | 90 | (26.3) | 12 | (3.5) |
| Decreased appetite | 87 | (25.7) | 7 | (2.1) | 79 | (23.1) | 3 | (0.9) |
| Platelet count decreased | 70 | (20.7) | 33 | (9.8) | 79 | (23.1) | 29 | (8.5) |
| Pyrexia | 68 | (20.1) | 5 | (1.5) | 56 | (16.4) | 2 | (0.6) |
| Vomiting | 62 | (18.3) | 5 | (1.5) | 62 | (18.1) | 7 | (2.0) |
| Diarrhea | 57 | (16.9) | 4 | (1.2) | 51 | (14.9) | 6 | (1.8) |
| Asthenia | 48 | (14.2) | 10 | (3.0) | 48 | (14.0) | 8 | (2.3) |
| Abdominal pain | 47 | (13.9) | 2 | (0.6) | 58 | (17.0) | 9 | (2.6) |
| Thrombocytopenia | 43 | (12.7) | 16 | (4.7) | 45 | (13.2) | 18 | (5.3) |
| Pruritis | 38 | (11.2) | — | | 28 | (8.2) | — | |
| Rash | 38 | (11.2) | 3 | (0.9) | 27 | (7.9) | 0 | |
| White blood cell count | 37 | (10.9) | 15 | (4.4) | 46 | (13.5) | 20 | (5.8) |
| decreased | | | | | | | | |
| Abdominal pain upper | 35 | (10.4) | 0 | | 30 | (8.8) | 1 | (0.3) |
| Insomnia | 32 | (9.5) | — | | 36 | (10.5) | — | |
| Cholangitis | 29 | (8.6) | 22 | (6.5) | 18 | (5.3) | 11 | (3.2) |
| Alanine aminotransferase | 29 | (8.6) | 4 | (1.2) | 35 | (10.2) | 2 | (0.6) |
| increased | | | | | | | | |
| Hypokalemia | 28 | (8.3) | 10 | (3.0) | 17 | (5.0) | 4 | (1.2) |
| Hyponatremia | 22 | (6.5) | 7 | (2.1) | 22 | (6.4) | 8 | (2.3) |
| Leukopenia | 20 | (5.9) | 8 | (2.4) | 17 | (5.0) | 3 | (0.9) |
| Hypertension | 20 | (5.9) | 6 | (1.8) | 20 | (5.8) | 7 | (2.0) |
| Pulmonary embolism | 16 | (4.7) | 8 | (2.4) | 13 | (3.8) | 7 | (2.0) |
| Sepsis | 15 | (4.4) | 12 | (3.6) | 9 | (2.6) | 8 | (2.3) |
| Biliary tract infection | 14 | (4.1) | 11 | (3.3) | 7 | (2.0) | 7 | (2.0) |
| Pneumonia | 14 | (4.1) | 9 | (2.7) | 10 | (2.9) | 6 | (1.8) |
| Acute kidney injury | 13 | (3.8) | 11 | (3.3) | 7 | (2.0) | 5 | (1.5) |
| Biliary obstruction | 11 | (3.3) | 7 | (2.1) | 10 | (2.9) | 8 | (2.3) |
| Blood bilirubin increased | 10 | (3.0) | 4 | (1.2) | 23 | (6.7) | 10 | (2.9) |

TABLE 17
Grade 3 or 4 Treatment-related Adverse Events That Occurred in ≥2%
of Patients in Either Treatment Group in the Safety Analysis Set.

| | Durvalumab plus Gemcitabine | | Placebo plus Gemcitabine | |
|---|---|---|---|---|
| | and Cisplatin | | and Cisplatin | |
| Adverse Events - no. (%) | (n = 338) | | (n = 342) | |
| Neutrophil count decreased | 70 | (20.7) | 87 | (25.4) |
| Neutropenia | 65 | (19.2) | 69 | (20.2) |
| Anemia | 64 | (18.9) | 64 | (18.7) |
| Platelet count decreased | 27 | (8.0) | 26 | (7.6) |
| White blood cell count | 14 | (4.1) | 20 | (5.8) |
| decreased | | | | |
| Thrombocytopenia | 12 | (3.6) | 18 | (5.3) |
| Fatigue | 9 | (2.7) | 8 | (2.3) |
| Leukopenia | 7 | (2.1) | 2 | (0.6) |
| Asthenia | 4 | (1.2) | 7 | (2.0) |

TABLE 18
Immune-mediated Adverse Events in the Safety Analysis Population.

| | Durvalumab plus | | | Placebo plus | |
|---|---|---|---|---|---|
| | Gemcitabine and | | | Gemcitabine and | |
| | Cisplatin (n = 338) | | | Cisplatin (n = 342) | |
| Adverse Events - no. (%) | Any Grade | | Grade 3 or 4 | Any Grade | Grade 3 or 4 |
| Any immune-mediated | 43 | (12.7) | 8 (2.4) | 16 (4.7) | 5 (1.5) |
| adverse event* | | | | | |
| Hypothyroid events | 20 | (5.9) | 0 | 5 (1.5) | 0 |
| Dermatitis/Rash | 12 | (3.6) | 3 (0.9) | 1 (0.3) | 0 |
| Pneumonitis | 3 | (0.9) | 1 (0.3) | 2 (0.6) | 1 (0.3) |
| Hepatic events | 4 | (1.2) | 2 (0.6) | 2 (0.6) | 1 (0.3) |
| Adrenal insufficiency | 4 | (1.2) | 0 | 1 (0.3) | 0 |
| Diarrhea/Colitis | 2 | (0.6) | 1 (0.3) | 1 (0.3) | 1 (0.3) |
| Hyperthyroid events | 2 | (0.6) | 0 | 0 | 0 |
| Type 1 diabetes mellitus | 1 | (0.3) | 1 (0.3) | 0 | 0 |
| Pancreatic events | 1 | (0.3) | 0 | 2 (0.6) | 1 (0.3) |
| Hypophysitis | 1 | (0.3) | 0 | 0 | 0 |
| Thyroiditis | 1 | (0.3) | 0 | 0 | 0 |
| Renal events | 0 | | 0 | 2 (0.6) | 0 |
| Myositis | 0 | | 0 | 1 (0.3) | 0 |
| Other rare/Miscellaneous† | 1 | (0.3) | 1 (0.3) | 1 (0.3) | 1 (0.3) |

*An immune-mediated adverse event is defined as an event that is associated with drug exposure and consistent with an immune-mediated mechanism of action and where there is no clear alternate etiology.
†The events in the “other rare/miscellaneous” category were immune-mediated arthritis in the durvalumab group and arthritis in the placebo group.

In previously untreated advanced biliary tract cancer, durvalumab plus gemcitabine and cisplatin demonstrated statistically significant prolonged overall survival versus placebo plus gemcitabine and cisplatin. The large, international patient population in TOPAZ-1 was representative of the general population of patients with advanced biliary tract cancer, and characteristics were well balanced between treatment groups. The addition of durvalumab to chemotherapy benefited patients with tumors characterized by PD-L1 TAP ≥1% and TAP <1%, demonstrating that PD-L1 status has limited value in predicting clinical benefit with durvalumab plus chemotherapy in this patient population. Durvalumab plus chemotherapy was associated with a safety profile and observed toxicities similar to those commonly seen with either chemotherapy or immunotherapy alone. Durvalumab did not add additional toxicity to that observed with chemotherapy in this double-blinded trial, and the rates of grade 3 or 4 treatment-related adverse events were very similar between treatment groups. The phase 3 TOPAZ-1 trial met the primary objective of improved overall survival at a preplanned interim analysis with durvalumab and chemotherapy while demonstrating manageable safety. Our global phase 3 trial showed a statistically significant, improvement in median overall survival with immunotherapy in advanced biliary tract cancer.

### BIBLIOGRAPHY

Aaronson et al., “The European Organization for Research and Treatment of Cancer QLQ-C30: A quality-of-life instrument for use in international clinical trials in oncology,” J. Natl. Cancer. Inst. 85 (5): 365-76 (1993).
Basch et al., “Adverse symptom event reporting by patients vs clinicians: relationships with clinical outcomes,” J. Natl. Cancer Inst. 101 (23): 1624-32 (2009).
Boilève et al., “Immunotherapy in Advanced Biliary Tract Cancers,” Cancers (Basel) 13 (7): 1569 (2021).
Bridgewater et al., “Biliary tract cancer: Epidemiology, radiotherapy, and molecular profiling,” Am. Soc. Clin. Oncol. Educ. Book 35: e194-203 (2016).
Brindley et al., “Cholangiocarcinoma,” Nat. Rev. Dis. Primers 7 (1): 65 (2021).
European Medicines Agency, “Guideline on the evaluation of anticancer medicinal products in man,” 2012 (accessed at www.ema.europa.eu/docs/en_GB/document_library/Scientific_guideline/2013/01/WC500137128.pdf).
EuroQol Group, “EQ-5D-5L user guide: Basic information on how to use the EQ-5D-5L instrument, version 2.1, April 2015,” 2015 (accessed Jun. 8, 2018 at euroqol.org/wp-content/uploads/2016/09/EQ-5D-5L_UserGuide_2015.pdf).
Fayers et al., “EORTC QLQ-C30 Scoring Manual” (3rd Edition 2001).
FDA-NIH Biomarker Working Group, “BEST (Biomarkers, EndpointS, and other Tools) Resource” (Silver Spring (MD): Food and Drug Administration (US), 2016) (accessed at www.ncbi.nlm.nih.gov/books/NBK338448).
Fife and Bluestone, “Control of peripheral T-cell tolerance and autoimmunity via the CTLA-4 and PD-1 pathways,” Immunol. Rev. 224:166-82 (2008).
Fiteni et al., “Cisplatin/gemcitabine or oxaliplatin/gemcitabine in the treatment of advanced biliary tract cancer: a systematic review,” Cancer Med. 3 (6): 1502-11 (2014).
Friend et al., “Development of a questionnaire (EORTC module) to measure quality of life in patients with cholangiocarcinoma and gallbladder cancer, the EORTC QLQ-BIL21,” Br. J. Cancer 104 (4): 587-92 (2011).
Gail and Simon, “Tests for qualitative interactions between treatment effects and patient subsets,” Biometrics 41 (2): 361-72 (1985).
Glimm et al., “Hierarchical testing of multiple endpoints in group-sequential trials,” Stat. Med. 29 (2): 219-28 (2010).
Kaup-Robberts et al., “Validation of the EORTC QLQ-BIL-21 questionnaire for measuring quality of life in patients with cholangiocarcinoma and cancer of the gallbladder,” Br. J. Cancer 115 (9): 1032-38 (2016).
Kim et al., “Incidence and overall survival of biliary tract cancers in South Korea from 2006 to 2015: Using the National Health Information Database,” Gut Liver 13 (1): 104-13 (2019).
Kluetz et al., “Incorporating the patient experience into regulatory decision making in the USA, Europe, and Canada,” Lancet Oncol. 19 (5): e267-74 (2018).
Lan and DeMets, “Discrete sequential boundaries for clinical trials,” Biometrika 70:659-63 (1983).
Lee et al., “Gemcitabine and oxaliplatin with or without erlotinib in advanced biliary-tract cancer: A multicentre, open-label, randomised, phase 3 study,” Lancet Oncol. 13 (2): 181-88 (2012).
Leone et al., “Panitumumab in combination with gemcitabine and oxaliplatin does not prolong survival in wild-type KRAS advanced biliary tract cancer: A randomized phase 2 trial (Vecti-BIL study),” Cancer 122 (4): 574-81 (2016).
Litwin et al., “Differences in urologist and patient assessments of health related quality of life in men with prostate cancer: Results of the CaPSURE database,” J. Urol. 159:1988-92 (1998).
Malka et al., “Gemcitabine and oxaliplatin with or without cetuximab in advanced biliary-tract cancer (BINGO): A randomised, open-label, non-comparative phase 2 trial,” Lancet Oncol. 15 (8): 819-28 (2014).
Okusaka et al., “Gemcitabine alone or in combination with cisplatin in patients with biliary tract cancer: A comparative multicentre study in Japan,” Br. J. Cancer 103 (4): 469-74 (2010).
Osoba et al., “Interpreting the significance of changes in health-related quality-of-life scores,” J. Clin. Oncol. 16 (1): 139-44 (1998).
Sprangers and Aaronson, “The role of health care providers and significant others in evaluating the quality of life of patients with chronic disease: A review,” J. Clin. Epidemiol. 45:743-60 (1992).
Sun and Chen, “Comparison of Finkelstein's Method with the conventional approach for interval-censored data analysis,” Stat. Biopharm. Res. 2 (1): 97-108 (2010).
Torre et al., “Worldwide burden of and trends in mortality from gallbladder and other biliary tract cancers,” Clin. Gastroenterol. Hepatol. 16 (3): 427-37 (2018).
Trask et al., “Patient-Reported Outcomes version of the Common Terminology Criteria for Adverse Events: Methods for item selection in industry sponsored oncology clinical trials,” Clin. Trials 15 (6): 616-23 (2018).
Tsiatis, “Repeated significance testing for a general class of statistics used in censored survival analysis,” J. Am. Stat. Assoc. 380:855-61 (1982).
U.S. Food and Drug Administration, “Guidance for Industry, Clinical Trial Endpoints for the Approval of Cancer Drugs and Biologics 2007,” 2007 (accessed at www.fda.gov/downloads/Drugs/Guidances/ucm071590.pdf).
U.S. Food and Drug Administration, “Summary of safety and effectiveness data,” 2017 (accessed Feb. 7, 2022, at www.accessdata.fda.gov/cdrh_docs/pdf17/P170019B.pdf).
Valle et al., “Cisplatin plus gemcitabine versus gemcitabine for biliary tract cancer,” N. Engl. J. Med. 362 (14): 1273-81 (2010).
Valle et al., “Cediranib or placebo in combination with cisplatin and gemcitabine chemotherapy for patients with advanced biliary tract cancer (ABC-03): A randomised phase 2 trial,” Lancet Oncol. 16 (8): 967-78 (2015).
Valle et al., “Biliary tract cancer,” Lancet 397 (10272): 428-44 (2021).
Valle et al., “Addition of ramucirumab or merestinib to standard first-line chemotherapy for locally advanced or metastatic biliary tract cancer: A randomised, double-blind, multicentre, phase 2 study,” Lancet Oncol. 22 (10): 1468-82 (2021).
Van Dyke et al., “Biliary tract cancer incidence and trends in the United States by demographic group, 1999-2013,” Cancer 125 (9): 1489-98 (2019).
van Hout et al., “Interim scoring for the EQ-5D-5L: Mapping the EQ-5D-5L to EQ-5D-3L value sets,” Value Health 15 (5): 708-15 (2012).
Wang et al., “The world-wide incidence of biliary tract cancer (BTC),”. J. Clin. Oncol. 38 (Suppl 4): 585 (2020).
Yachida et al., “Genomic Sequencing Identifies ELF3 as a Driver of Ampullary Carcinoma,” Cancer Cell 29:229-40 (2016).

### EMBODIMENTS

Embodiment 1: A method of treating a patient with biliary tract cancer (BTC), the method comprising treating the patient with an anti-PD-L1 antibody and chemotherapy.
Embodiment 2: A method for increasing overall survival in a patient with biliary tract cancer (BTC), the method comprising treating the patient with an anti-PD-L1 antibody and chemotherapy.
Embodiment 3: A method for extending progression-free survival in a patient with biliary tract cancer (BTC), the method comprising treating the patient with an anti-PD-L1 antibody and chemotherapy.
Embodiment 4: An anti-PD-L1 antibody in combination with chemotherapy for use in the treatment of biliary tract cancer (BTC).
Embodiment 5: An anti-PD-L1 antibody in combination with chemotherapy for use in the treatment of biliary tract cancer (BTC) in a patient, wherein overall survival is increased in said patient.
Embodiment 6: An anti-PD-L1 antibody in combination with chemotherapy for use in the treatment of biliary tract cancer (BTC) in a patient, wherein progression-free survival is extended in said patient.
Embodiment 7: The use of an anti-PD-L1 antibody in combination with chemotherapy in the manufacture of a medicament for use in the treatment of biliary tract cancer (BTC).
Embodiment 8: The use of an anti-PD-L1 antibody in combination with chemotherapy in the manufacture of a medicament for use in the treatment of biliary tract cancer (BTC) in a patient, wherein overall survival is increased in said patient.
Embodiment 9: The use of an anti-PD-L1 antibody in combination with chemotherapy in the manufacture of a medicament for use in the treatment of biliary tract cancer (BTC) in a patient, wherein progression-free survival is extended in said patient.
Embodiment 10: The method, combination for use or use of any one of embodiments 1-9, wherein the chemotherapy comprises concurrent chemotherapy.
Embodiment 11: The method, combination for use or use of any one of embodiments 1-10, wherein the chemotherapy comprises a platinum-based chemotherapy and/or an antimetabolite-based chemotherapy.
Embodiment 12: The method, combination for use or use of embodiment 11, wherein the platinum-based chemotherapy is cisplatin or carboplatin.
Embodiment 13: The method, combination for use or use of embodiment 11 or embodiment 12, wherein the antimetabolite-based chemotherapy is gemcitabine.
Embodiment 14: The method, combination for use or use of any one of embodiments 1-13, wherein the anti-PD-L1 antibody is durvalumab, avelumab, or atezolizumab.
Embodiment 15: The method, combination for use or use of any one of embodiments 1-14, wherein the anti-PD-L1 antibody and/or chemotherapy is administered intravenously.
Embodiment 16: The method, combination for use or use of any one of embodiments 1-15, wherein the anti-PD-L1 antibody is administered to the patient every three weeks (Q3W).
Embodiment 17: The method, combination for use or use of any one of embodiments 1-16, wherein the anti-PD-L1 antibody and a dose of chemotherapy are administered concurrently or consecutively.
Embodiment 18: The method, combination for use or use of any one of embodiments 1-17, wherein a second dose of chemotherapy is administered 7 days after the administration of a first dose of chemotherapy.
Embodiment 19: The method, combination for use or use of any one of embodiments 1-18 comprising a treatment cycle lasting 21 days, wherein:
(a) the anti-PD-L1 antibody and the first dose of chemotherapy are administered on day 1 of the treatment cycle, and
(b) the second dose of chemotherapy is administered on day 8 of the treatment cycle.
Embodiment 20: The method, combination for use or use of embodiment 19, where the treatment cycle is repeated 1, 2, 3, 4, 5, 6, 7, or 8 times.
Embodiment 21: The method, combination for use or use of any one of embodiments 1-20, wherein the anti-PD-L1 antibody is administered after the completion of the treatment with chemotherapy.
Embodiment 22: The method, combination for use or use of any one of embodiments 19-21, wherein the anti-PD-L1 antibody is administered after the end of the final treatment cycle.
Embodiment 23: The method, combination for use or use of embodiment 22, wherein the anti-PD-L1 antibody is administered once every four weeks (Q4W).
Embodiment 24: The method, combination for use or use of any one of embodiments 1-23, wherein treatment with the human anti-PD-L1 antibody comprises administering 1000-2000 mg of the anti-PD-L1 antibody.
Embodiment 25: The method, combination for use or use of embodiment 24, wherein treatment with the human anti-PD-L1 antibody comprises administering 1500 mg of the anti-PD-L1 antibody.
Embodiment 26: The method, combination for use or use of any one of embodiments 1-25, wherein treatment with chemotherapy comprises administering gemcitabine 1000 mg/m² and cisplatin 25 mg/m² to the patient intravenously.
Embodiment 27: The method, combination for use or use of any one of embodiments 1-26, wherein the biliary tract cancer is unresectable, locally advanced, or metastatic.
Embodiment 28: The method, combination for use or use of any one of embodiments 1-27, wherein the patient has not been previously treated for BTC.
Embodiment 29: The method, combination for use or use of any one of embodiments 1-28, wherein the patient developed recurrent disease more than 6 months after surgery with curative intent or more than 6 months after the completion of adjuvant therapy.
Embodiment 30: The method, combination for use or use of any one of embodiments 1-29, wherein observed toxicities are similar to those commonly seen with either chemotherapy or immunotherapy alone.

What is claimed is:

1. A method for increasing overall survival in a patient with biliary tract cancer (BTC), the method comprising treating the patient with an anti-PD-L1 antibody and chemotherapy.

2. The method of claim 1, wherein the chemotherapy comprises concurrent chemotherapy.

3. The method of either claim 1 or claim 2, wherein the chemotherapy comprises a platinum-based chemotherapy and/or an antimetabolite-based chemotherapy.

4. The method of claim 3, wherein the platinum-based chemotherapy is cisplatin or carboplatin.

5. The method of claim 3, wherein the antimetabolite-based chemotherapy is gemcitabine.

6. The method of any one of claims 1-5, wherein the anti-PD-L1 antibody is durvalumab, avelumab, sugemalimab or atezolizumab.

7. The method of any one of claims 1-6, wherein the anti-PD-L1 antibody and/or chemotherapy is administered intravenously.

8. The method of any one of claims 1-7, wherein the anti-PD-L1 antibody is administered to the patient every three weeks (Q3W).

9. The method of any one of claims 1-8, wherein the anti-PD-L1 antibody and a dose of chemotherapy are administered concurrently or consecutively.

10. The method of any one of claims 1-9, wherein a second dose of chemotherapy is administered 7 days after the administration of a first dose of chemotherapy.

11. The method of any one of claims 1-10 comprising a treatment cycle lasting 21 days, wherein:
(a) the anti-PD-L1 antibody and the first dose of chemotherapy are administered on day 1 of the treatment cycle, and
(b) the second dose of chemotherapy is administered on day 8 of the treatment cycle.

12. The method of claim 11, where the treatment cycle is repeated 1, 2, 3, 4, 5, 6, 7, or 8 times.

13. The method of any one of claims 1-12, wherein the anti-PD-L1 antibody is administered after the completion of the treatment with chemotherapy.

14. The method of any one of claims 11-13, wherein the anti-PD-L1 antibody is administered after the end of the final treatment cycle.

15. The method of claim 14, wherein the anti-PD-L1 antibody is administered once every four weeks (Q4W).

16. The method of any one of claims 1-15, wherein treatment with the human anti-PD-L1 antibody comprises administering 1000-2000 mg of the anti-PD-L1 antibody.

17. The method of claim 16, wherein treatment with the human anti-PD-L1 antibody comprises administering 1500 mg of the anti-PD-L1 antibody.

18. The method of any one of claims 1-17, wherein treatment with chemotherapy comprises administering gemcitabine 1000 mg/m² and cisplatin 25 mg/m² to the patient intravenously.

19. The method of any one of claims 1-18, wherein the biliary tract cancer is unresectable, locally advanced, or metastatic.

20. The method of any one of claims 1-19, wherein the patient has not been previously treated for BTC.

21. The method of any one of claims 1-20, wherein the patient developed recurrent disease more than 6 months after surgery with curative intent or more than 6 months after the completion of adjuvant therapy.

22. The method of any one of claims 1-21, wherein observed toxicities are similar to those commonly seen with either chemotherapy or immunotherapy alone.

23. A method for extending progression-free survival in a patient with biliary tract cancer (BTC), the method comprising treating the patient with an anti-PD-L1 antibody and chemotherapy.

24. The method of claim 23, wherein the chemotherapy comprises concurrent chemotherapy.

25. The method of either claim 23 or claim 24, wherein the chemotherapy comprises a platinum-based chemotherapy and/or an antimetabolite-based chemotherapy.

26. The method of claim 25, wherein the platinum-based chemotherapy is cisplatin or carboplatin.

27. The method of claim 25, wherein the antimetabolite-based chemotherapy is gemcitabine.

28. The method of any one of claims 23-27, wherein the anti-PD-L1 antibody is durvalumab, avelumab, sugemalimab or atezolizumab.

29. The method of any one of claims 23-28, wherein the anti-PD-L1 antibody and/or chemotherapy is administered intravenously.

30. The method of any one of claims 23-29, wherein the anti-PD-L1 antibody is administered to the patient every three weeks (Q3W).

31. The method of any one of claims 23-30, wherein the anti-PD-L1 antibody and a dose of chemotherapy are administered concurrently or consecutively.

32. The method of any one of claims 23-31, wherein a second dose of chemotherapy is administered 7 days after the administration of a first dose of chemotherapy.

33. The method of any one of claims 23-32 comprising a treatment cycle lasting 21 days, wherein:
(a) the anti-PD-L1 antibody and the first dose of chemotherapy are administered on day 1 of the treatment cycle, and
(b) the second dose of chemotherapy is administered on day 8 of the treatment cycle.

34. The method of claim 11, where the treatment cycle is repeated 1, 2, 3, 4, 5, 6, 7, or 8 times.

35. The method of any one of claims 23-33, wherein the anti-PD-L1 antibody is administered after the completion of the treatment with chemotherapy.

36. The method of any one of claims 23-35, wherein the anti-PD-L1 antibody is administered after the end of the final treatment cycle.

37. The method of claim 36, wherein the anti-PD-L1 antibody is administered once every four weeks (Q4W).

38. The method of any one of claims 32-37, wherein treatment with the human anti-PD-L1 antibody comprises administering 1000-2000 mg of the anti-PD-L1 antibody.

39. The method of claim 38, wherein treatment with the human anti-PD-L1 antibody comprises administering 1500 mg of the anti-PD-L1 antibody.

40. The method of any one of claims 23-39, wherein treatment with chemotherapy comprises administering gemcitabine 1000 mg/m² and cisplatin 25 mg/m² to the patient intravenously.

41. The method of any one of claims 23-40, wherein the biliary tract cancer is unresectable, locally advanced, or metastatic.

42. The method of any one of claims 23-41, wherein the patient has not been previously treated for BTC.

43. The method of any one of claims 23-42, wherein the patient developed recurrent disease more than 6 months after surgery with curative intent or more than 6 months after the completion of adjuvant therapy.

44. The method of any one of claims 23-43, wherein observed toxicities are similar to those commonly seen with either chemotherapy or immunotherapy alone.

45. A method of treating a patient with biliary tract cancer (BTC), the method comprising treating the patient with an anti-PD-L1 antibody and chemotherapy.

46. The method of claim 45, wherein the chemotherapy comprises concurrent chemotherapy.

47. The method of either claim 45 or claim 46, wherein the chemotherapy comprises a platinum-based chemotherapy and/or an antimetabolite-based chemotherapy.

48. The method of claim 47, wherein the platinum-based chemotherapy is cisplatin or carboplatin.

49. The method of claim 47, wherein the antimetabolite-based chemotherapy is gemcitabine.

50. The method of any one of claims 45-49, wherein the anti-PD-L1 antibody is durvalumab, avelumab, sugemalimab or atezolizumab.

51. The method of any one of claims 45-50, wherein the anti-PD-L1 antibody and/or chemotherapy is administered intravenously.

52. The method of any one of claims 45-51, wherein the anti-PD-L1 antibody is administered to the patient every three weeks (Q3W).

53. The method of any one of claims 45-52, wherein the anti-PD-L1 antibody and a dose of chemotherapy are administered concurrently or consecutively.

54. The method of any one of claims 45-53, wherein a second dose of chemotherapy is administered 7 days after the administration of a first dose of chemotherapy.

55. The method of any one of claims 45-54 comprising a treatment cycle lasting 21 days, wherein:
(a) the anti-PD-L1 antibody and the first dose of chemotherapy are administered on day 1 of the treatment cycle; and
(b) the second dose of chemotherapy is administered on day 8 of the treatment cycle.

56. The method of claim 55, where the treatment cycle is repeated 1, 2, 3, 4, 5, 6, 7, or 8 times.

57. The method of any one of claims 45-56, wherein the anti-PD-L1 antibody is administered after the completion of the treatment with chemotherapy.

58. The method of any one of claims 45-57, wherein the anti-PD-L1 antibody is administered after the end of the final treatment cycle.

59. The method of claim 58, wherein the anti-PD-L1 antibody is administered once every four weeks (Q4W).

60. The method of any one of claims 45-59, wherein treatment with the human anti-PD-L1 antibody comprises administering 1000-2000 mg of the anti-PD-L1 antibody.

61. The method of claim 60, wherein treatment with the human anti-PD-L1 antibody comprises administering 1500 mg of the anti-PD-L1 antibody.

62. The method of any one of claims 45-61, wherein treatment with chemotherapy comprising administering gemcitabine 1000 mg/m² and cisplatin 25 mg/m² to the patient intravenously.

63. The method of any one of claims 45-62, wherein the biliary tract cancer is unresectable, locally advanced, or metastatic.

64. The method of any one of claims 45-63, wherein the patient has not been previously treated for BTC.

65. The method of any one of claims 45-64, wherein the patient developed recurrent disease more than 6 months after surgery with curative intent or more than 6 months after the completion of adjuvant therapy.

66. The method of any one of claims 45-65, wherein observed toxicities are similar to those commonly seen with either chemotherapy or immunotherapy alone.

67. An anti-PD-L1 antibody in combination with chemotherapy for use in the treatment of biliary tract cancer (BTC).

68. An anti-PD-L1 antibody in combination with chemotherapy for use in the treatment of biliary tract cancer (BTC) in a patient, wherein overall survival is increased in said patient.

69. An anti-PD-L1 antibody in combination with chemotherapy for use in a method of increasing overall survival in a patient with biliary tract cancer (BTC).

70. An anti-PD-L1 antibody in combination with chemotherapy for use in the treatment of biliary tract cancer (BTC) in a patient, wherein progression-free survival is extended in said patient.

71. An anti-PD-L1 antibody in combination with chemotherapy for use in a method of extending progression-free survival in a patient with biliary tract cancer (BTC).

72. Use of an anti-PD-L1 antibody in combination with chemotherapy in the manufacture of a medicament for use in the treatment of biliary tract cancer (BTC).

73. Use of an anti-PD-L1 antibody in combination with chemotherapy in the manufacture of a medicament for use in a method of increasing overall survival in a patient with biliary tract cancer (BTC).

74. Use of an anti-PD-L1 antibody in combination with chemotherapy in the manufacture of a medicament for use in a method of extending progression-free survival in a patient with biliary tract cancer (BTC).
